(19) United States
(12) Patent Application Publication
(10) Pub. No.: US 2020/0155840 A1
(43) Pub. Date: May 21, 2020

(54) SYSTEM FOR AIDING EARLY DETECTION AND MANAGEMENT OF BREATHING DISORERS

(71) Applicant: Health Apps Pty Ltd, Kew , Victoria (AU)

(72) Inventors: John GIANNOUKOS, Templestowe, Victoria (AU); Arthur KORFIATIS, Balwyn North Victoria (AU)

(21) Appl. No.: 16/631,858

(22) PCT Filed: Jul. 17, 2018
(86) PCT No.: PCT/AU2018/005751

§ 371 Date: Jan. 17, 2020

(30) Foreign Application Priority Data
Jul. 17, 2017 (AU) 2017902791

(51) Int. Cl. A61N 1/36; A61B 5/00; A61N 1/04; A61B 5/0205; A61B 5/11; A61B 5/1455

(52) U.S. Cl. CPC A61N 1/3601; A61B 5/024; A61N 1/0456; A61N 1/0452; A61N 1/36031; A61B 5/02055; A61B 5/11; A61B 5/14551; A61B 5/7405; A61B 5/742; A61B 5/7455; A61B 5/0002; A61N 1/36034; A61B 2503/04; A61B 5/4818

(57) ABSTRACT

A system for aiding early detection and management of breathing related disorders including a plurality of the at least one external sensor and a control means for receiving to the predefined operative framework a predefined effective operative range of each of the plurality of the at least one external sensor; wherein sensing by at least two of the plurality of the at least one external sensor with a trigger analysis including reviewing multiple sensors and if multiple single sensor trigger points from multiple sensors have been received within a predefined time period there is created a trigger actuation and outputting trigger output to operative framework to instigate external stimulation and if multiple single sensor trigger points from multiple sensors have not been received within a predefined time period there is a return to further sensing and further trigger analysis.

### FIELD OF THE INVENTION

The invention relates to a system for aiding early detection and management of breathing related disorders and in particular to a system for aiding management of disorders such as sleep apnea (apnea) or snoring or sudden infant death syndrome (SIDS) or infant apnea.

The invention has been developed primarily for use in/with health industry and self-help health industry for aiding assessing and/or pre-treating of sleep apnea and will be described hereinafter with reference to this application.

It will be appreciated that the invention is not limited to this particular field of use.

### BACKGROUND OF THE INVENTION

A system for aiding early detection and management of breathing related disorders is usually a treatment that is implemented after the event of the disorder has fully developed. This is the case when managing snoring or sleep apnea. In other breathing related disorders, it is possible that there is no pre-warning or normal symptoms and therefore often fatalities occur.

Millions of people around the world are dependent on continuous positive airway pressure (CPAP) machines to help them manage snoring and sleep apnea. CPAP devices deliver a stream of compressed air via a hose and mask, attempting to keeping the airway open under air pressure, and enabling improved/unobstructed breathing.

CPAP machines are cumbersome, bulky, noisy and can in themselves be the cause of awakening people with Sleep Apnea. The success of a CPAP machine is reliant on correct fitting, avoiding airleaks and continued use of cumbersome fitout of tubes. They can also be detrimental or aid to complications in people with heart disease or who have suffered a stroke. In some people CPAP machines prevent restful sleep and their use is associated with high blood pressure, arrhythmia, stroke and heart failure. Heart disease is the leading cause of death in the United States, and stroke is also a leading cause of death and disability. It therefore cannot be considered to be the solution for all cases and alternatives are needed. A large percentage of people stop using CPAP machines in first few months.

The trouble with these problems is that millions of people around the world are dependent on continuous positive airway pressure (CPAP) machines to help them manage snoring and sleep apnea. Also, it is important in most medical fields to have preventative measures rather than treatment measures.

It can be seen that breathing related disorders have one or more of the problems of:
a) Minor breathing or non-breathing interruptions;
b) Breathing disorder effects such as snoring,
c) Breathing difficulties which is emphasized by startling reflexes, smothering or other direct physical effect;
d) Uncomfortableness while sleeping due to variable breathing resulting in heat and oxygen variation where not wanted
e) Indirect effects such as lack of oxygen circulation;
f) Dire consequences such as lack of oxygen to the brain;
g) the inappropriateness of internal aids; or
h) risks associated with surgical procedures.

It is known to have sleep apnea treatment systems. One form is a CPAP machine. Some of the inconveniences of a CPAP machine include:
It's intrusive and cumbersome to wear the mask.
Difficult to get a good night's sleep as the mask, air tubes, air leaks, pump noise continuously awakens you.
Monitoring and assessment is not easy or straight forward, usually requires medical personal to assist and to analyse any data the CPAP machine may collect.
Once a CPAP machine has been recommended for users, there has been a missed opportunity to diagnose and manage at an earlier stage.

It is known to have systems which are implanted into the patient. These have the inconveniences of:
a) Expensive surgery;
b) Very invasive;
c) Not readily changeable;
d) Expensive and therefore not available to most;
e) Only used in very critical cases due to expense and therefore not available for prevention or early diagnosis of critical cases;
f) Inability to upgrade hardware without additional surgery;
g) If implant solution does not offer expected results—additional surgery for removal.

It can be seen that known techniques for treating breathing related disorders have a range of problems and the present invention seeks to provide a system for aiding management of breathing related disorders, which will overcome or substantially ameliorate at least one or more of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

According to the invention there is provided a system for aiding early detection and management of a breathing related disorder comprising: at least one sensor for sensing and providing sensed data of at least one characteristic relevant to a breathing related disorder; a predefined operative framework having connection for receiving the sensed data; at least one external stimulator positionable relative to a user and operatively connectable with the predefined operative framework for providing an effective external stimulation to the user; a control means for providing to the predefined operative framework a predefined effective operative range of the at least one external stimulator; wherein the predefined effective treatment range and control aids early detection and management of a breathing related disorder by an effective external stimulation.

The breathing related disorders can include one or more of:
Sleep apnea
Snoring
SIDS (Sudden Infant Death Syndrome)

Preferably the effective external stimulation by the at least one external stimulator effects an alteration to the user's breathing.

The effective external stimulation by the at least one external stimulator can be by indirectly effecting an alteration to the user's breathing. Such indirectly effecting of an alteration to the user's breathing can be by notification to the user.

The system for aiding early detection and management of breathing related disorders can have the effective external stimulation by the at least one external stimulator substantially directly effecting an alteration to the user's breathing. Such directly effecting of an alteration to the user's breathing can be selected from one or more of:
an external stimulator locatable on or near the ear of the user providing acoustic output for stimulation to the user,
an external stimulator locatable under chin area for stimulation to the user,
an external stimulator locatable on the head for external stimulation to the user,
an external stimulator locatable on the finger or limbs for stimulation to the user,
an external stimulator using TENS (Transcutaneous Electrical Nerve Stimulation) or EMS (Electrical Muscle Stimulation) or both locatable to provide for stimulation to the user, or
an external stimulator locatable on the foot for stimulation to the user.
wherein the external stimulators can operate in a mode that directly affects a user to improve the breathing of the user.

Most preferably the communication between sensors, control and at least one 20 external stimulator is wired and/or wireless.

According to an aspect of the present invention, a system for aiding early detection and management of breathing related disorders is provided by an external stimulation of the tongue in order to improve openness of the airway.

It can be seen that the invention provides a system for the benefit of allowing ready non-invasive preventative, early management, and management tool for aiding management of breathing related disorders.

The invention in one particularly advantageous form has a system for aiding early detection and management of breathing related disorders including a plurality of the at least one external sensor and a control means for receiving to the predefined operative framework a predefined effective operative range of each of the plurality of the at least one external sensor; wherein sensing by at least two of the plurality of the at least one external sensor within the predefined effective treatment range and control aids early detection and management of a breathing related disorder by an effective sensing by the at least one sensor.

The control means can include a collaborator and each sensor receives sensed data and assesses if a predefined trigger point has been sensed and sends identification of single sensor trigger point to the collaborator so it can receive and undertake a trigger analysis including reviewing multiple sensors. If multiple single sensor trigger points from multiple sensors have been received within a predefined time period there is created a trigger actuation and outputting trigger output to operative framework to instigate external stimulation. If multiple single sensor trigger points from multiple sensors have not been received within a predefined time period there is a return to further sensing and further trigger analysis.

In this way the recordal of a single sensor trigger point of one sensor does not initiate trigger actuation unless supported by a single sensor trigger point of another sensor.

In a particular preferred form of multiple sensors the system for aiding early detection and management of breathing related disorders has the user controller including connection with one or more input selectable sensor modules providing the at least one sensor. The one or more input selectable modules includes a selected sensor internal module part for connection with the user controller and an external sensor module part for location on an external body location and for providing sensed data to the selected sensor internal module part.

Also in a preferred form there is provided a system for aiding early detection and management of breathing related disorders having the user controller including connection with one or more output selectable stimulator modules providing the at least one external stimulator. The one or more output selectable stimulator modules includes a selected stimulator internal module part for connection with the user controller and an external stimulator module part for location on an external body location and for providing stimulation to the selected stimulator external module part.

It can be seen that the invention of the system for aiding early detection and management of breathing related disorders provides the benefit of ready selection and connection of different sensors and different stimulators so that a particularly suitable and effective and acceptable system and device is provided for that particular user as per the user's choice, the medical practitioner's advice on breathing and sleeping disorders, the carer's choice or a combination thereof.

According to another aspect of the present invention, a system for aiding early detection and management of breathing related disorders is provided by there being provided an external stimulation of the hypoglossal nerve region and surrounding area around the submental tongue area in order to improve openness of the airway

It can be seen that the invention of the system for aiding early detection and management of breathing related disorders provides the benefit of research which showed that the best way to stimulate the tongue is to stimulate the hypoglossal nerve/region, around the submental area. This in turn causes the tongue to contract based on level of stimulation set by user.

According to a further aspect of the present invention, a system for aiding early detection and management of breathing related disorders is provided by a variable external stimulation of the tongue in order to retain effectiveness of improving openness of the airway.

It can be seen that the invention of the system for aiding early detection and management of breathing related disorders provides the benefit of research which showed that the best way to continue to stimulate the tongue is to change the stimulation of the hypoglossal nerve/region, around the submental area. In this way the nerve does not become immune or numbed to the stimulation but is kept reactive and readily useable as an effective stimulant to the tongue to cause contraction and open the airway.

In one form the invention provides a system a system for aiding management of a breathing related disorder comprising at least one sensor for sensing a characteristic of a breathing related disorder, an access port for upload from the sensor to an online computerised means adapted for following computerised and automated instructions according to a predefined effective treatment range in managing control of a breathing related disorder; at least one topical stimulator positionable on a user for providing an effective stimulation option to the user and including a receiver for receiving electronic control instructions; at least one input device for allowing a user to predefine at least one user particular stimulation output and provide for upload to the access port; at least one display device for allowing display of an external stimulation output to the user to provide a confirmed user defined management control of the breathing related disorder application; at least one transmitter for transmitting each user particular input of the plurality of users to the access port of the online means for upload and transfer of data with the online computerised means; and at least one receiver for transmitting for receiving by at least one of the at least one display device for allowing display of the respective confirmed user defined breathing related disorder application and functions of each user; wherein the plurality of user particular inputs defines the predefined effective treatment range and control of a breathing related disorder and the selection of an effective stimulation option defines the chosen effective option and is provided to the at least one display device; wherein the system for aiding early detection and management of breathing related disorders can be substantially assembled with improving effectiveness in aiding early detection and management of breathing related disorders including any one or more of the following:
i. improvements in structure and assembly including ease of operation in order to allow ready external use;
ii. Improvements in stimulation including better operation for self-use;
iii. Improvements in control of variation of stimulation by providing predefined options and selection techniques;
iv. is easy to use by people and control the unit using their smart device such as computers, smart phones, health fitness wrist bands, tablets and watches, with interacting via cable or wireless including WiFi, Bluetooth or other wireless technologies.
v. The Health App comes with endless opportunities to manage awareness, inform family, medical support people or any support or carer person for activities to other health manage plans to assess overall outcomes, etc and
vi. The Health App updates will be easily applied onto the user's device or manually as needed by user.

It can be seen that the invention of a system for aiding early detection and management of breathing related disorders provides the benefit of non-invasive preventative and management tool of aiding management of disorders such as sleep apnea or snoring or sudden infant death syndrome (SIDS).

The invention can deliver a small portable solution that is simple to use, enables people to better assess and manage their apnea/snoring condition and delivers improved overall early management, significantly reducing the risk of related chronic disorders as people are more likely to continue using a more comfortable solution at an early stage and potentially delay moving to a CPAP machine is significantly cheaper than surgical options without associated risks.

The invention can provide multiple combination of external stimulations and notifications either concurrently, sequentially or as selected.

Further benefits of this system are that it is easy to use, does not require professional and/or medical support to use, is significantly cheaper than a CPAP machine and delays the transition to a CPAP machine.

Other aspects of the invention are also disclosed.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 is a general diagrammatic view of a system for aiding management of a breathing related disorder for use in the health industry and self-help health industry in accordance with a preferred embodiment of the present invention;

FIG. 2 is a diagrammatic view of multiple sensors with primary sensors and secondary sensors for use in an embodiment of the system of FIG. 1;

FIGS. 3, 4 and 5 are diagrammatic views of user controller with choice of a selection of sensor modules and details of the internal sensor module part and external sensor module part;

FIG. 6 is a diagrammatic view of the selection of categories of location of sensors that can be for use in the system for aiding management of a breathing related disorder of FIG. 1;

FIG. 7 is a diagrammatic view of the selection of categories of mode of operation of sensors that can be for use in the system for aiding management of a breathing related disorder of FIG. 1;

FIG. 8 is an explanatory cutaway of a detail of the hypoglossal nerves that can be activated externally from the throat to improve breathing in system for aiding management of a breathing related disorder of FIG. 1;

FIGS. 9, 10 and 11 are diagrammatic views of user controller with choice of a selection of external stimulator modules and details of the internal stimulator module part and external stimulator module part;

FIG. 12 is a diagrammatic view of the selection of categories of location of stimulators that can be for use in the system for aiding management of a breathing related disorder of FIG. 1;

FIG. 13 is a diagrammatic view of the selection of categories of mode of operation of stimulators that can be for use in the system for aiding management of a breathing related disorder of FIG. 1;

FIG. 14 is a diagrammatic block diagram of the operation of the sensors of the system for aiding management of a breathing related disorder of FIG. 1;

FIG. 15 is a diagrammatic block diagram of the operation of multiple of the sensors of the system for aiding management of a breathing related disorder of FIG. 1;

FIG. 16 is a diagrammatic view of the timing operation of trigger actuation by multiple of the sensors of the system for aiding management of a breathing related disorder of FIG. 1;

FIG. 17 is a diagrammatic block diagram of the operation of the external stimulator of the system for aiding management of a breathing related disorder of FIG. 1;

FIG. 18 is an overall diagrammatic view of a range of functional options of the system for aiding management of a breathing related disorder of FIG. 1;

FIG. 19 is a diagrammatic view of the self-modifying operation of the sensors and external stimulators by feedback and control in a variable system for aiding management of a breathing related disorder of FIG. 1;

FIG. 20 is a diagrammatic view of the self-changing control of the sensor usable in the self-modifying operation of the sensors and external stimulators by feedback and control in a variable system for aiding management of a breathing related disorder of FIG. 1;

FIG. 21 is an operative flow diagram of the operation of the self-changing operation of the sensors of FIG. 20;

FIG. 22 is a diagrammatic view of the self-changing control of the external stimulator usable in the self-modifying operation of the sensors and external stimulators by feedback and control in a variable system for aiding management of a breathing related disorder of FIG. 1; and

FIG. 23 is an operative flow diagram of the operation of the self-changing operation of the external stimulators of FIG. 22.

### DESCRIPTION OF PREFERRED EMBODIMENTS

It should be noted in the following description that like or the same reference numerals in different embodiments denote the same or similar features.

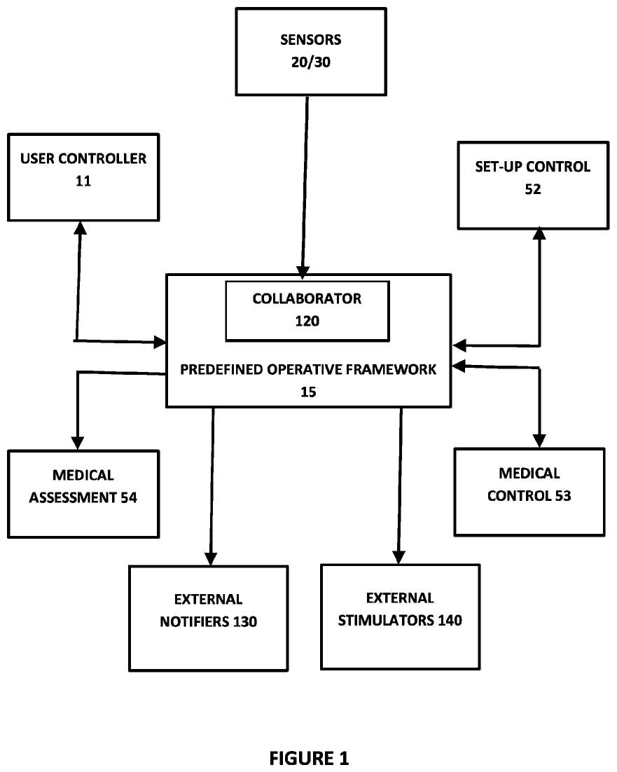

Referring to the drawings, in FIG. 1 there is shown a general application of a system for aiding early detection and management of a breathing related disorder in accordance with an embodiment of the invention. The system for a user has at least one sensor 20/30 from a plurality of sensors 20 for sensing at least one characteristic of a breathing related disorder. The breathing disorder can relate to breathing related disorders such as sleep apnea, snoring or even SIDS (Sudden Infant Death Syndrome).

There is provided a predefined operative framework 15 that is in receivable connection to the sensors 20 to receive information from the sensors about the sensed characteristic. The predefined operative framework 15 further has operable connection to at least one external stimulator of a plurality of stimulators 140 positionable relative to a user for providing an effective external stimulation option to the user.

The predefined operative framework 15 is connected or connectable to a range of other connections including the user controller for the user to be able to make input and assist control. In this regard the user controller 11 could be a separate device to the predefined operative controller 15 such as a mobile telephone connecting to a modem having the predefined operative controller 15. In another form the predefined operative controller 15 can be integral, insertable or downloadable into the user controller 11.

Apart from the predefined operative controller 15 being connectable to receive the user's input there can be a set-up control 52 that can be remote and able to be downloaded or integral or insertable into the user controller 11 or predefined operative controller 15. This allows for set-ups of sensors 20 and stimulators 140 according to their characteristics and according to set-up rules. The user controller 11 or predefined operative controller 15 is also connected or connectable to medical control 53 so that a medical practitioner can affect input controls according to medical practice, particularity of the particular user as a medical patient, or due to developments of medical and technical understanding of the operation of the sensors and stimulators in providing aids early detection and management of a breathing related disorder. An output of the medical assessment 54 can provide sensed data to the medical practitioner the user or other carers for use in aiding early detection and management of a breathing related disorder. There also can be simple external notifiers as triggers or advice of a detection by the device or system so that carers or those related to users are advised of sensed data or trigger points for use in aiding early detection and management of a breathing related disorder.

As will be further described the ability for selectable choice or change of sensors or change of operation of sensors and the selectable choice or change of stimulators or change of operation of stimulators is important in allowing this system to be adaptable to the most effective form for the user and to retain effectiveness as the user acclimatises or builds up a resistance to the single operation of use.

The system and device for aiding management of a breathing related disorder also acts as a gatherer of sensed data for use by medical practitioners for precise diagnosis rather than the self-use that is an aid to diagnosis, maintenance and treatment. Clearly though a user cannot be under 24-hour medical supervision seven days a week so a system to support and augment the relationship between user and medical practitioner is clearly advantageous. Also, the system and device provide a substantial minimization of oversight of indicators as this is dramatically reduced by the extended use of the aid by the user.

There is also a control means for providing to the at least one topical stimulator a predefined effective treatment range of the at least one topical stimulator positionable on the user in managing control of a breathing related disorder of the user. The control means can be to an external online computerised means or to an app on a user's smart telecommunication device or a combination.

The control means can provide a predefined effective treatment range of the one external stimulator positionable relative to a user, which control aids management of the breathing related disorder by an effective external stimulation or notification.

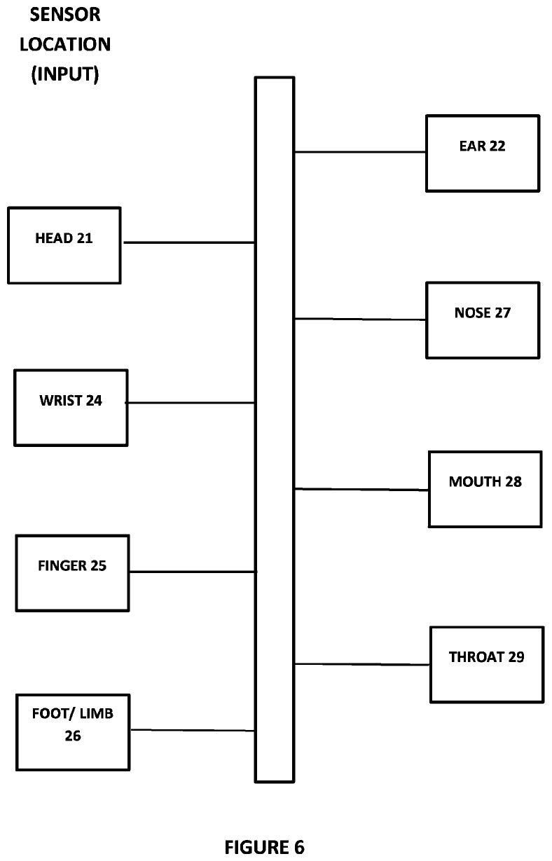

As shown in FIG. 6 the sensors 20 can be selected from one or more locations such as sensor 21 locatable on the head of the user, sensor 22 locatable near mouth or nose or lungs for detecting breath of user, sensor 23 locatable on the ear, sensor 24 locatable on the wrist, sensor 25 locatable on the finger or sensor 26 locatable on the foot and can be sensing characteristic that is relevant to the breathing of the user. The sensors 20 can operate in a mode that senses a particular characteristic that is relevant to the breathing of the user.

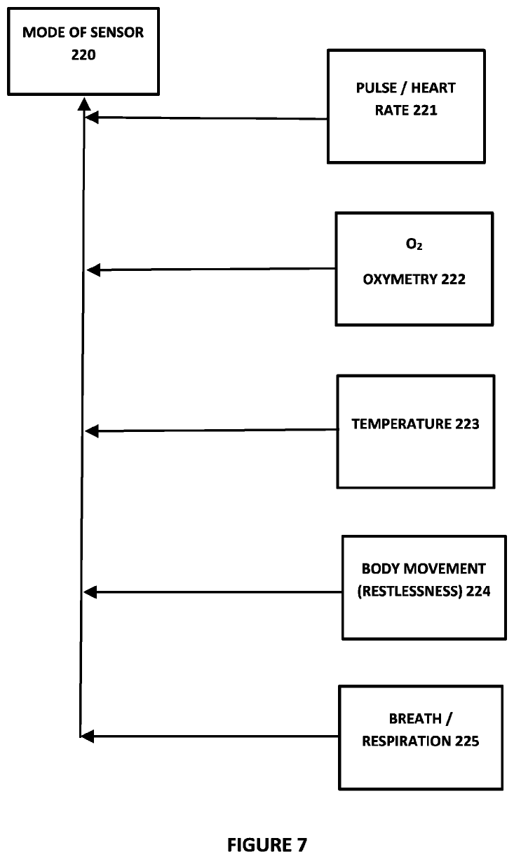

Referring to FIG. 7, the various modes 220 of sensor can be a mode 221 to detect the characteristic of the pulse/heart rate, a mode 222 to detect the characteristic of oxygen levels, a mode 223 to detect the characteristic of temperature of the user, or a mode 224 to detect the characteristic of body movement or restlessness of the user and their settings. All of these modes 220 have a different operating range within which sensing will indicate a particular breathing effectiveness. Changes of the characteristic out of those ranges or sudden changes in that range can indicate prewarning issues of the breathing related disorders.

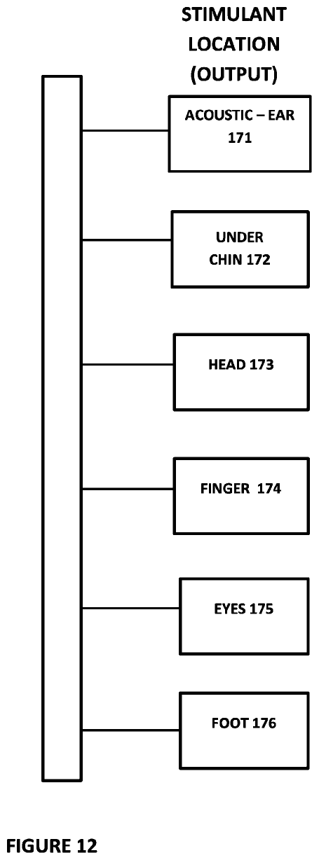

As shown in FIG. 12 the stimulators 140 can be selected from one or more locations such as external stimulator 41 locatable on or near the ear of the user providing acoustic output for stimulation or notification to the user, external stimulator 42 locatable under chin for stimulation or notification to the user, external stimulator 43 locatable on the head for external stimulation or notification to the user, external stimulator 44 locatable on the finger for stimulation or notification to the user, external stimulator 45 using TENS (Transcutaneous Electrical Nerve Stimulation) or EMS (Electrical Muscle Stimulation) or both locatable to provide for stimulation or notification to the user, or external stimulator 46 locatable on the foot for stimulation or notification to the user. The external stimulators 40 can operate in a mode that affects a user to improve the breathing of the user.

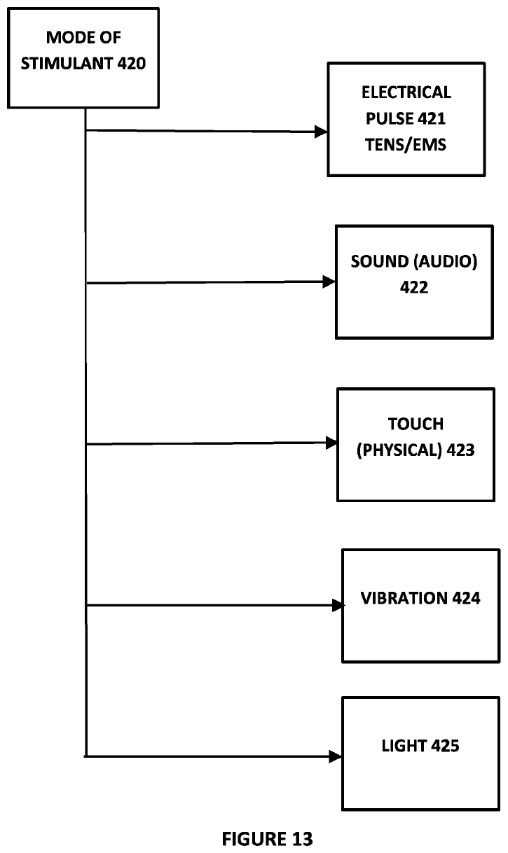

Referring to FIG. 13, the various modes 420 of external stimulators can be a mode 421 to provide electrical pulse or stimulation through TENS and/or EMS, a mode 422 to provide the external stimulation or notification by sound (audio), a mode 423 to provide the external stimulation or notification by touch (physical), or a mode 424 to provide the external stimulation or notification by vibration applied to the user. All of these modes 420 have a different operating range within which external stimulation or notification will improve a particular breathing effectiveness. Changes of the characteristic out of those ranges or sudden changes in that range can indicate prewarning issues of the breathing related disorders.

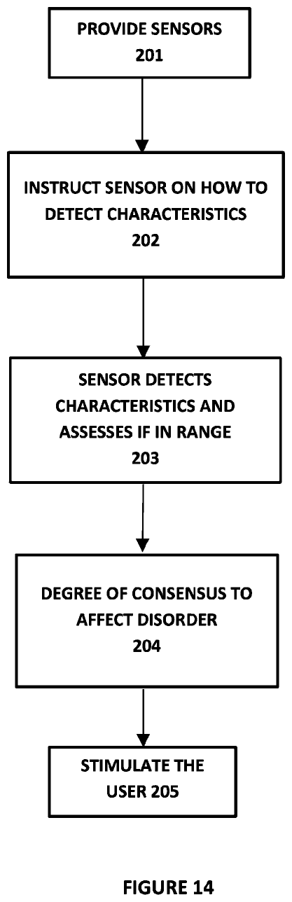

Referring to FIG. 14 there is shown a partial operation of the system of FIG. 1 in which in a first step 201 the selection of sensors 20 are provided. This allows a user to select one or more sensors 20 and allow connection to the predefined operative framework 15. Such framework can be provided on a smart device solely or in combination with a remote computer or cloud-based platform connected by wireless telecommunication.

In a second step 202 the predefined operative framework 15 which is operatively connected to the selected one or more of the sensors 20 and to the selected one or more of the external stimulators 140 and requires instructions on how to detect characteristics of the particular user using the particular sensor for the particular characteristics relevant to the breathing disorder. In effect, the sensors themselves do not need to be particularly pre-programmed and specialised for the purpose but can be smart devices that are set-up by the predefined operative framework 15. This can be achieved by such framework having predefined controls therein and/or by communication with a plurality of controls.

The controls can be a user control 51 such that a user can enter sensor product information and personal dimension information of relevance such as gender, weight, preconditions, condition of concern and other details. There also can be a medical control 53 that interprets the personal information and the expected breathing related disorders and provides a framework for the characteristic relevant to the breathing disorder. A third control can be a set-up control 52 that is determinative of the activating controls and sensitivities and calibration details of the selected sensor which allows for the specialised adaption in situ to the operation of the system.

As shown in Step 203 the sensor 20 detects the relevant characteristics relevant to the breathing disorder and assesses if in range as determined by the medical control 53. This can further include wireless communication to remote computer or cloud-based platform for such review or be maintained in a comparative review on the user's smart device.

In step 204 there is the review of the degree of consensus that would affect the disorder. In this regard, a false alarm is not beneficial and could stimulate when not required and cause stress to the user which exacerbates the situation rather than assists the situation. Therefore, the consensus can be to see the length of time that a sensor detects characteristics assessed in the relevant range or undertaking further sensing and if three sensed readings detected in the assessed range then consensus with the sensed characteristics does warrant action by the stimulator or notificator to the user in order to aid early detection and management of the breathing related disorder.

Thereby in Step 205 the external stimulator is activated according to the controls 51, 52 and 53 within a predefined effective treatment range of the one external stimulator positionable relative to a user, and which aids management of the breathing related disorder by an effective external stimulation or notification.

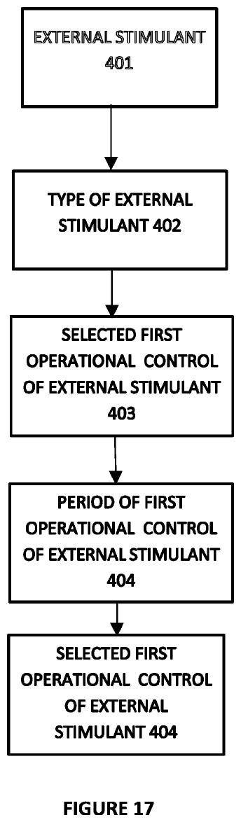

Referring to FIG. 17 there is shown a partial operation of the system of FIG. 1 in which in a first step 401 the selection of external stimulators 40 are provided. This allows a user to select one or more external stimulators 40 and allow connection to the predefined operative framework 15. Such framework can be provided on a smart device solely or in combination with a remote computer or cloud-based platform connected by wireless telecommunication.

In a second step 402 the predefined operative framework 15 which is operatively connected to the selected one or more of the external stimulators 40 and requires instructions on how to provide the effective stimulation or notification to the user to effect the response required to improve relevant breathing disorder. In effect, the external stimulators 40 themselves do not need to be particularly pre-programmed and specialised for the purpose but can be smart devices that are set-up by the predefined operative framework 15. This can be achieved by such framework having predefined controls therein and/or by communication with a plurality of controls.

In step 403 there is the selected first operational control of the external stimulant by the controls can be the user control 51 such that a user can enter external stimulator product information and personal dimension information of relevance such as gender, weight, preconditions, condition of concern and other details. There also can be the medical control 53 that interprets the personal information and the expected breathing related disorders and provides a framework for the determined treatment stimulation or notification relevant to assess, pre-warn or improve the breathing disorder. A third control can be the set-up control 52 that is determinative of the activating controls and sensitivities and calibration details of the selected external stimulators which allows for the specialised adaption in situ to the operation of the system.

In step 404 there is the determination of the periodic time of stimulation and/or the length of operation of the external stimulator 140. This can be in reference to the user control 51, set-up control 52 and medical control 53.

Then by Step 405 the at least one external stimulator 140 positionable relative to a user and operatively connectable with the predefined operative framework for provides an effective external stimulation to the user within a predefined effective operative range of the at least one external stimulator and wherein the predefined effective treatment range and control aids early detection and management of a breathing related disorder by an effective external stimulation.

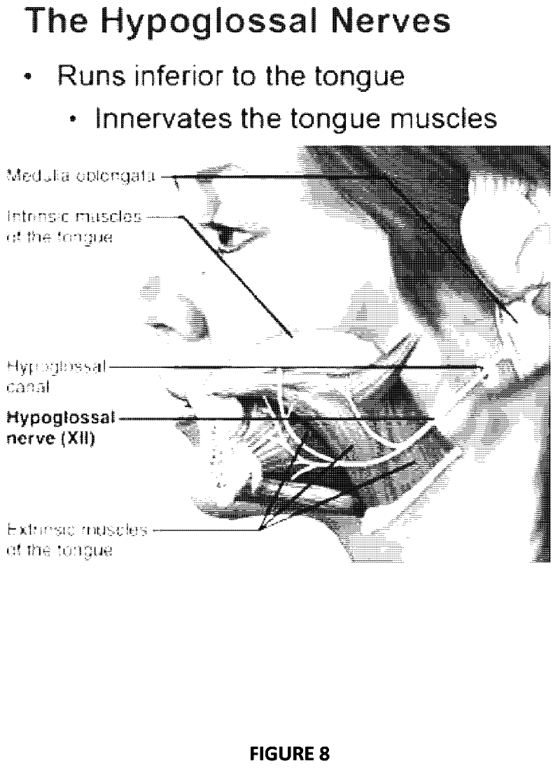

In one embodiment of a particular anatomical external stimulation with reference to the anatomical drawing of FIG. 8, there is provided an external stimulation of the tongue in order to improve openness of the airway and effect improved breathing or reduce snoring or assist other breathing related disorders. In another form, the invention includes an external stimulation of the hypoglossal nerve region around the submental area tongue in order to improve openness of the airway. In still another form the invention there is provided a variable external stimulation of the tongue in order to retain effectiveness of improving openness of the airway.

As shown in FIGS. 6, 7 and 18, there is shown the sensors and location and mode of the sensors and the stimulant and mode and location of the stimulant that can be enacted based on the sensor. These stimulants and location can include acoustic stimulant located at or near the ear, touch or acoustic or electric pulse stimulant at or near the chin, head, finger foot. In a particularly preferred form that is believed beneficial is an external stimulant to the hypoglossal nerve using a TENS system.

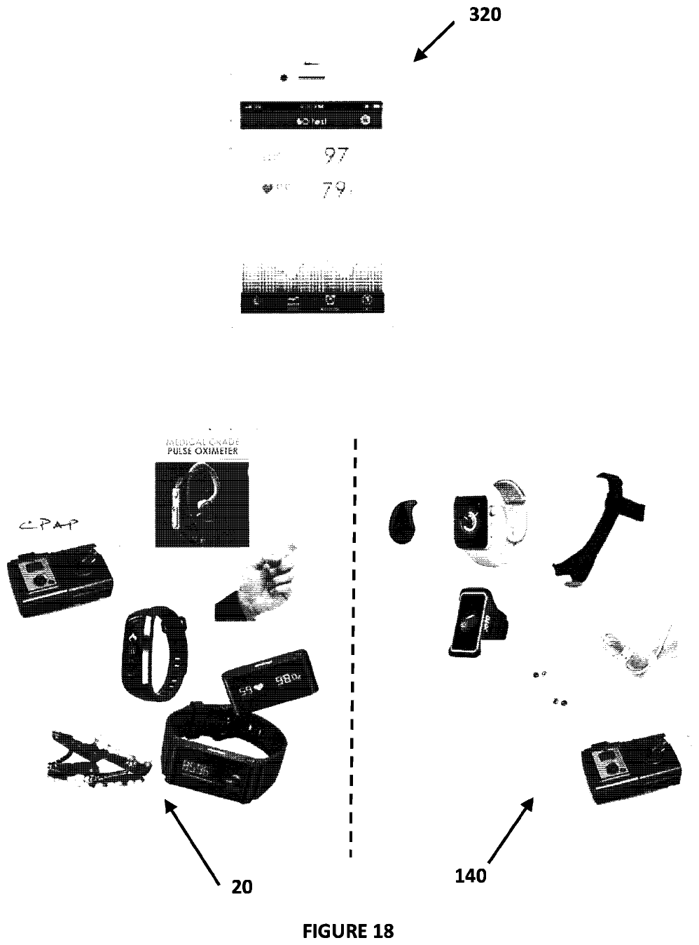

As shown in FIG. 18 In a particular preferred form, there is provided a range of technologies that can be combined into this system to allow for aiding management of a breathing related disorder which includes at least one sensor for sensing at least one characteristic of a breathing related disorder; a predefined operative framework having at least one external stimulator positionable relative to a user for providing an effective external stimulation option to the user a control means for providing to the at least one topical stimulator a predefined effective treatment range of the at least one topical stimulator positionable on the user in managing control of a breathing related disorder of the user; wherein the predefined effective treatment range and control aids management of a breathing related disorder by an effective external stimulation.

Multiple Sensors

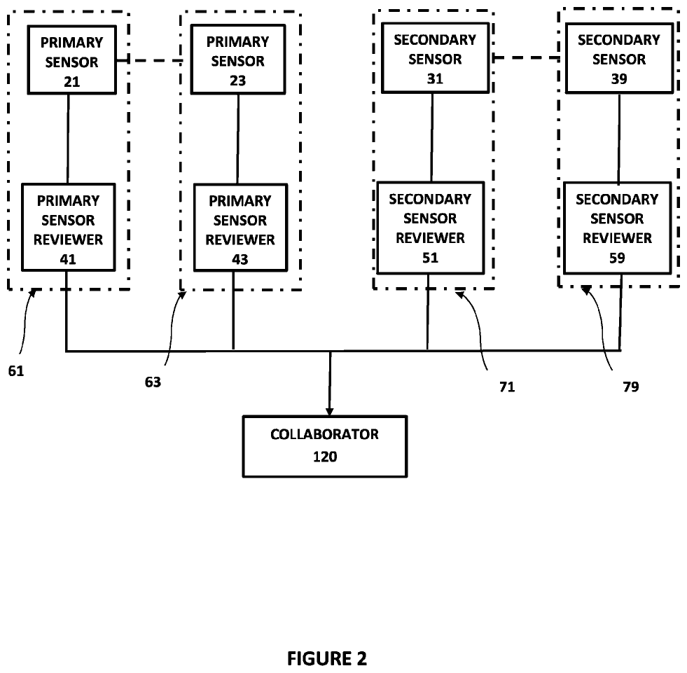

Referring to FIG. 2 there is shown the use of multiple sensors. This in one advantageous form includes a primary sensor 20 and a secondary sensor 30. In this way multiple sensors deliver uplifted accuracy even if the primary sensor is the more accurate or detailed or more suitable for the user. However, the invention allows the ability to maintain only an external sensing of the user rather than the invasiveness prior art having a device with invasive internal sensors, which can only be used under strict medical or professional supervision. This prior art immediately detracted from the wish for the user to use the device and will then increase the risk of ill effects of breathing related disorder because there is less detection. Therefore, the present system ensures by the use of external sensors that no detraction occurs and the user is extremely willing to use it to over extended times.

The complication with external sensors is that there are increases in false positive readings in that sensed data could incorrectly advise of sensed data that would be expected to indicate an occurrence of ill effect of a breathing related disorder. If the stimulator is then applied the user is affected or woken or alarmed for incorrect reasonings. It is therefore particularly beneficial to have a primary sensor 21 with its secondary sensor reviewer 41 as well as a secondary sensor 31 with its secondary sensor reviewer 51.

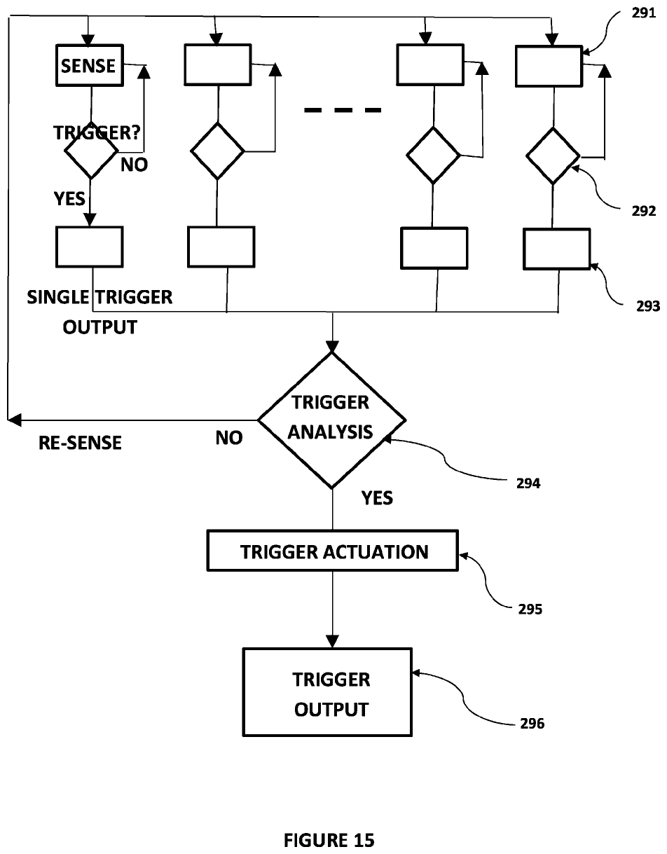

The first effect therefore as shown in FIG. 2 is that the primary sensor 21 has its sensed data reviewed by the primary sensor reviewer 41 and if the reviewer automatically assesses and compares the sensed data with operating conditions that should indicate an ill effect then a single sensor trigger provides a single sensor trigger output to the collaborator 120 as part of the operative framework 15. This does not yet result in a trigger as shown in FIG. 15 but instead needs to be supported by a further sensor.

Due to the difference in effectiveness of sensors it is considered that they would be categorized into primary and secondary sensors and that in the plurality of sensors used there is at least one primary sensor.

Looking at FIG. 15 there is a sensing at step 291 by each of the plurality of sensors used and if in the single sensor review at step 292 there is automatic assessment that the sensed data falls outside acceptable levels then the reviewer at step 293 issues the single trigger output. At step 294 the collaborator receives multiple sensed data or not and if over a particular time period receives two or more single

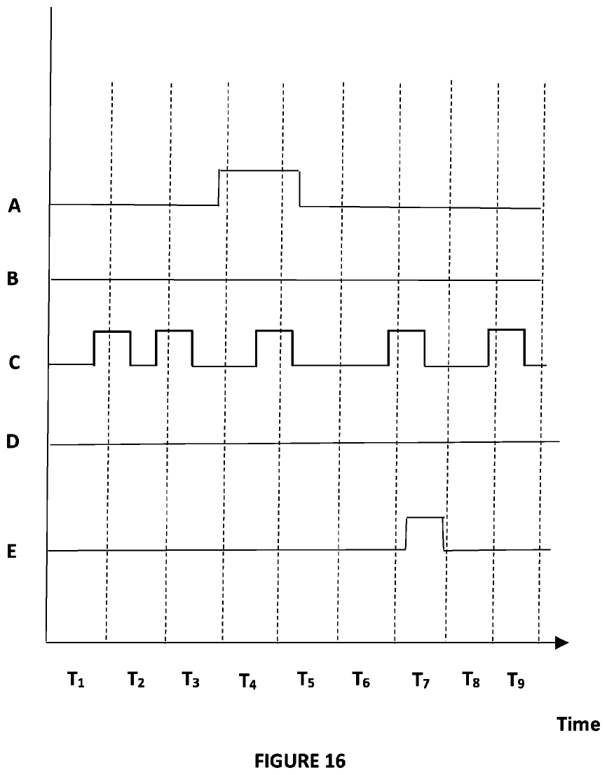

FIG. 16 is illustrative at the time period receipt of multiple signals or not from the single sensors. Clearly if sensor C was the only sensor then the user would receive 5 alarms over the period of T1 to T9 but it is expected that a number of those are false positives. By having Sensor C with Sensor A then it is only during T4 that there is two single sensor triggers and therefore trigger actuation step 295 will only occur at T4.

Similarly as shown if sensor B or D was used then no

Primary sensors can be one of the following:
O2 Oxygen. (Levels below 92% Oxygen in your blood is a sign of a breathing problem.
Pulse/Heart Rate
Blood pressure

Secondary sensors can be:
Geo-Positioning (Upright, Laying down, Moving Activity)
Temperature
Diaphragm movement.
Steps & Activity & Floors Climbed
Calories Burned
Sleep Tracking
Sleep Stages (Light, Deep, REM)

Referring to Pulse Oximetry Overview there is a direct correlation between Oxygen levels and Heart rate for Apnea related chronic conditions. This can also be extended to people suffering from snoring.

You will also see pulse oximetry being used in a number of clinical settings, such as in the emergency department and in the operating theatre, to assess and monitor patents, so it is important to know about its uses and limitations, as well as how to interpret the readings that it produces.

The fundamental principle behind pulse oximetry is that when you shine light of a certain wavelength at molecules of oxygenated and deoxygenated haemoglobin differing amounts of light are absorbed by these molecules. So, if you place a light source emitting these specific wavelengths of light on one side of the finger and a sensor that detects these wavelengths of light on the other side, one can measure the amount of light being absorbed within the tissue by oxygenated and deoxygenate haemoglobin. The reading that is produced (the SpO2) represents the percentage of oxygenated haemoglobin present as a proportion of the total amount of haemoglobin detected. So, a reading of 92% means that the pulse oximeter has detected that 92% of the haemoglobin molecules sampled are carrying oxygen and 8% are deoxygenated molecules. Pulse oximeters are designed to provide readings on haemoglobin molecules that are travelling in a pulsatile manner, so the reading represents the situation that exists in the arterial circulation. You will see pulse oximetry probes being applied to fingers and toes and also to hands, feet and earlobes in babies

It can be that the user or more particularly the user under review by a medical practitioner, completes a Questionnaire to obtain a high-level snapshot of user's position. This can be used to assess various risk rating(s) and recommend number of sensors to use.

The key input functions can have readings which can be used in any combination by the user as part of their monitoring configuration, which is fully configurable by the user or by their medical practitioner.

By the importance of Sensor C being a primary sensor, if it is used in combination with a secondary sensor B or D and those sensors failed to pick up any trigger point by the time the C sensor has triggered 3 times then the 3^{rd} trigger of the primary sensor advises of the multiple single sensor trigger and warns for testing or changing of the secondary sensors.

The great benefit of the system is that Sensor B and D which might be defective or not suitable or effective for the particular user can readily be replaced by connection to different sensor such as Sensor A or E and thereby the system again functions to its maximum effectiveness while eliminating false negatives.

Apart from the idea that the second sensor would need to have sensed data that in itself would be reviewed by its sensor reviewer to determine if trigger for ill effect, and that therefore there are two triggers and avoidance of false negatives, a substantial benefit of the plurality of sensors is that the sensor can be operating with less sensitivity and still be effective in combination. In particular the pick-up of the sensor can be operating at 80% but is amplified. This in effect increases the chance of false negatives by a single sensor but that is offset by the need for the second or multiple sensors to trigger to provide an actual trigger event. This provides greater overall accuracy from external non-invasive sensors without having to use more accurate or more intrusive or internal sensors.

In effect therefore the plurality of sensors allows a plurality of the at least one external sensor and a control means for receiving to the predefined operative framework a predefined effective operative range of each of the plurality of the at least one external sensor; wherein sensing by at least two of the plurality of the at least one external sensor within the predefined effective treatment range and control aids early detection and management of a breathing related disorder by an effective sensing by the at least one sensor.

Modules

A particular advantageous version of the invention includes the use of modules in order for the user, the medical professional or advisor to select and connect modules that are particularly effective to you.

1. Input Selectable Sensor Modules

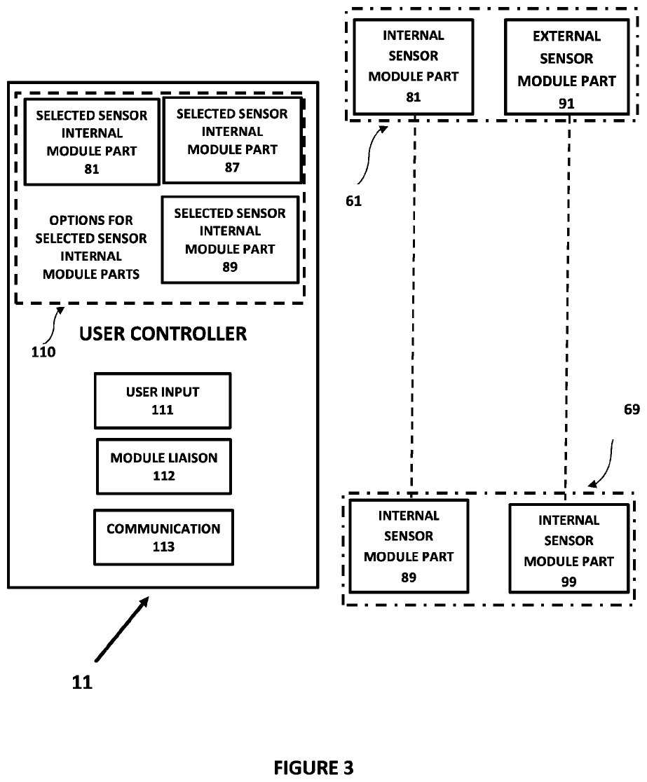

Referring to FIGS. 3, 4 and 5 there is shown an embodiment of a system for aiding early detection and management of breathing related disorders wherein the user controller includes connection with one or more input selectable sensor modules providing the at least one sensor.

The user controller 11 has a user input 111 for receiving input of selection or usage requirements from the user. However the primary input is from sensors and has the user controller 11 having location 110, for options for receipt physically or digitally of selected sensor internal module parts.

The user can select a sensor 61 to 69 and each sensor will have its internal sensor module part 81 to 89 and corresponding external sensor module part 91. These parts can be integral but generally will be in wireless communication.

The one or more input selectable modules includes a selected sensor internal module part for connection with the user controller and an external sensor module part for location on an external body location and for providing sensed data to the selected sensor internal module part.

The internal module parts 81, 87, 89 of the selected sensors 61, 67, 69 will be downloadable or insertable into the user controller and can communicate through the module liaison 112 as required with the rest of the operative framework 15 of the user controller 11 or with external connections through the communication 113.

The external sensor module part 91 to 99 is positionable to sense at at least one or more of the following external body sensor locations of:
Head
throat
nose
mouth
Ear
Wrist
Finger
Foot/limb

The external sensor module part is selected to sense according to at least one of the following modes of:
Pulse/heart rate
Oximetry/O2 levels
Temperature
Body movement
breath

It can therefore be that a user (which can be under the guidance of a medical practitioner) undertakes a selection of primary sensor module 61 and secondary sensor module 71 and these are installed into the user controller 11 with an internal part 81 in communication with an external part 91 that is attached externally to the user when in use.

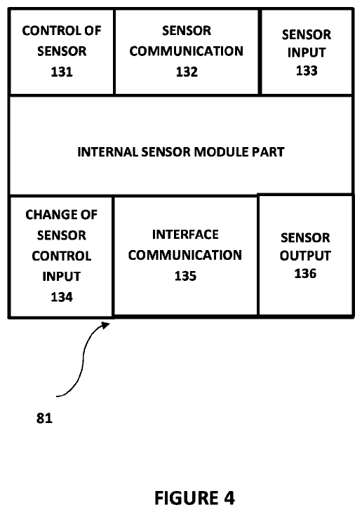

The internal sensor module part 81, as shown in FIG. 4 includes the primary element of the sensor input 133 for receiving from the corresponding external sensor module part 91 which is detecting the required characteristic at the required location and under the required mode. This communication can be directly between the internal and external parts or through the communication interface 135 to the communication part 113 of the user controller.

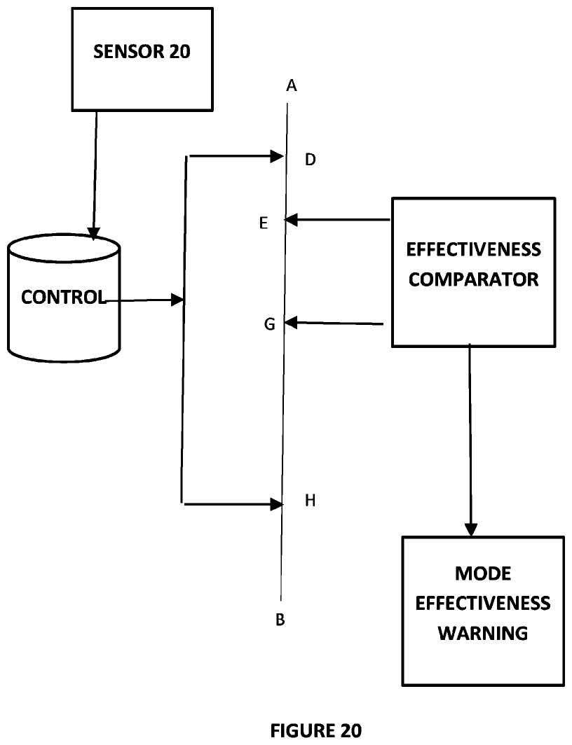

The internal sensor module part 81 further includes a control of sensor part 131 for providing the required operating settings and control mechanisms of the external sensor module part 91. This control section 131 can include inherent controls that are predefined in order to operate the sensor and can receive changes, elaborations, calibrations or fine tuning from a change of sensor control input 134. This input can receive input directly through user input 111 of the user controller from the user or directly or indirectly from an external set-up control 52 such as downloadable from an updatable external website. There also can be input directly from medical control 53 such as updates on effectiveness of particular sensing of characteristics for indicating particular breathing disorders or adjustments by the medical practitioner due to the actual patient characteristics or requirements. Further the change of sensor control input 134 can receive feedback control information such as in FIG. 20 so that the operation of the sensor can be self-calibrated. This is of particular benefit due to the external use of sensor and variability of sensor sensitivity in that form of use.

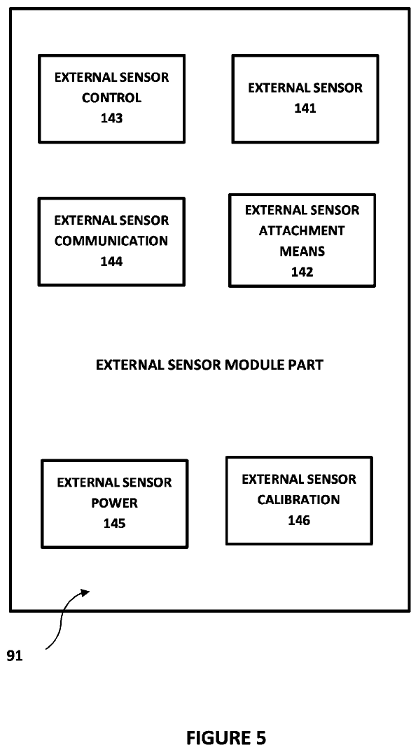

The external sensor module part 91 of FIG. 5 also has its component parts including the primary external sensor 141 that is placed in the required position with the required mode. This can be retained in place by the external sensor attachment means 142 that could be a strap, belt, adhesive, or other attachment means.

The external sensor 141 can be controlled by the external sensor control 143 that can be in communication with the internal sensor communication module through the external sensor communication 144. Preferably this is wireless so as to not limit the positioning of the external sensor module part 91 with the user controller but could have wired connection or be integral. The external module 91 has an external sensor power 145 that can be due to battery, wireless power, wired power, solar, movement or otherwise powered. Further the operation of the external sensor can be calibrated by its direct sensing and review or through the further processing done separately from the external module such as in the user controller, in the internal sensor module part or further afield in the remote website.

It can be seen that having sensor modules and particularly with the incorporation of internal sensor module parts and external sensor module parts the device is particularly effective in providing a more effective application of a plurality of external sensors without wires extending everywhere and with coordination with single user controller while still being networked for medical assessment and medical control.

2. Output Selectable Stimulator Modules

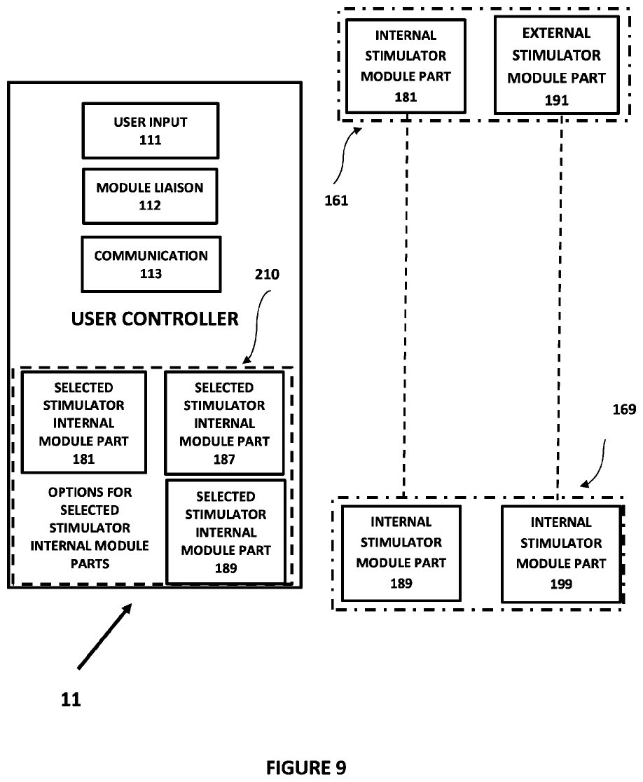

Referring to FIGS. 9, 10 and 11 there is shown an embodiment of a system for aiding early detection and management of breathing related disorders wherein the user controller includes connection with one or more output selectable stimulator modules providing the at least one external stimulator.

The one or more output selectable stimulator modules includes a selected stimulator internal module part for connection with the user controller and an external stimulator module part for location on an external body location and for providing stimulation to the selected stimulator external module part.

The user controller 11 has a user input 111 for receiving input of selection or usage requirements from the user. However the primary input is from sensors and has the user controller 11 having location 110, for options for receipt physically or digitally of selected sensor internal module parts.

The user can select a stimulator 181 to 189 and each sensor will have its internal sensor module part 191 to 199 and corresponding external sensor module part 161. These parts can be integral but generally will be in wireless communication.

The one or more input selectable modules includes a selected stimulator internal module part for connection with the user controller and an external stimulator module part for location on an external body location and for providing stimulation via the selected stimulation external module part.

The internal module parts 181, 187, 189 of the selected stimulators 161, 167, 169 will be downloadable or insertable into the user controller and can communicate through the module liaison 112 as required with the rest of the operative framework 15 of the user controller 11 or with external connections through the communication 113.

The external stimulator module part 191 to 199 is positionable to stimulate at at least one or more of the following external body sensor locations of:
Head
throat
nose
mouth
Ear
Wrist
Finger
Foot/limb

The external stimulator module part 191 to 199 is selected to sense according to at least one of the following modes of:
Electrical pulse TENS/EMS
Sound/Audio
Touch/Physical
Vibration
Light
Other party notification
(SMS/text alert message or integral app.)

It can therefore be that a user (which can be under the guidance of a medical practitioner) undertakes a selection of stimulation modules 181 and these are installed into the user controller 11 with an internal part 181 in communication with an external part 191 that is attached externally to the user when in use.

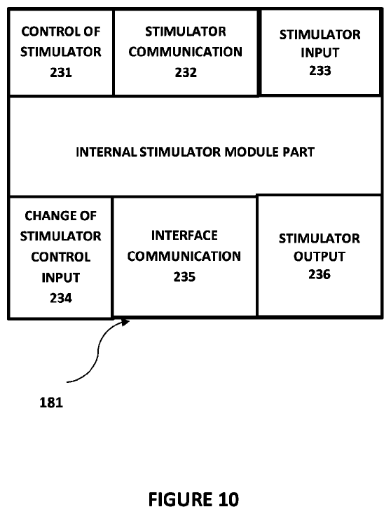

The internal stimulation module part 181, as shown in FIG. 10 includes the primary element of the stimulation input 233 for receiving from the corresponding external stimulation module part 191 which is detecting the required characteristic at the required location and under the required mode. This communication can be directly between the internal and external parts or through the communication interface 235 to the communication part 213 of the user controller.

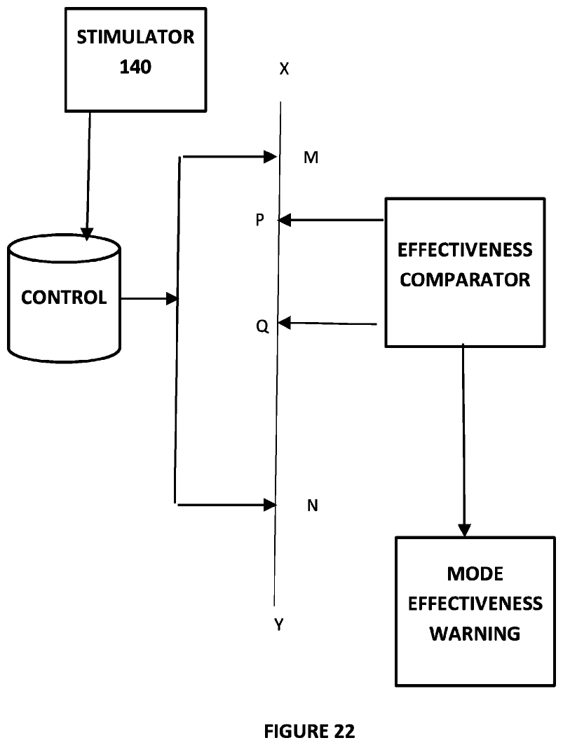

The internal stimulation module part 181 further includes a control of stimulation part 231 for providing the required operating settings and control mechanisms of the external stimulation module part 191. This control section 231 can include inherent controls that are predefined in order to operate the stimulation and can receive changes, elaborations, calibrations or fine tuning from a change of s stimulation control input 234. This input can receive input directly through user input 111 of the user controller from the user or directly or indirectly from an external set-up control 52 such as downloadable from an updatable external website. There also can be input directly from medical control 53 such as updates on effectiveness of particular stimulation of characteristics for indicating particular breathing disorders or adjustments by the medical practitioner due to the actual patient characteristics or requirements. Further the change of stimulation control input 234 can receive feedback control information such as in FIG. 22 so that the operation of the stimulation can be self-calibrated. This is of particular benefit due to the external use of stimulation and variability of stimulation sensitivity in that form of use.

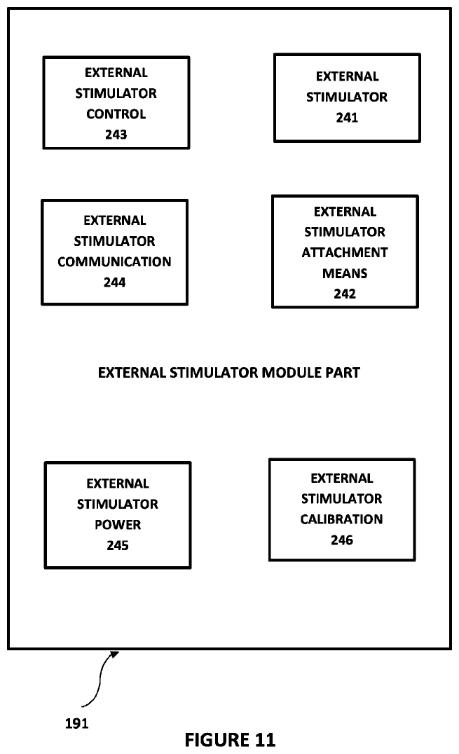

The external stimulation module part 191 of FIG. 11 also has its component parts including the primary external stimulation 241 that is placed in the required position with the required mode. This can be retained in place by the external stimulation attachment means 242 that could be a strap, belt, adhesive, or other attachment means.

The external stimulation 241 can be controlled by the external stimulation control 243 that can be in communication with the internal stimulation communication module through the external stimulation communication 244. Preferably this is wireless so as to not limit the positioning of the external stimulation module part 191 with the user controller but could have wired connection or be integral. The external module 191 has an external stimulation power 245 that can be due to battery, wireless power, wired power, solar, movement or otherwise powered. Further the operation of the external stimulation can be calibrated by its direct sensing and review or through the further processing done separately from the external module such as in the user controller, in the internal stimulation module part or further afield in the remote website.

It can be seen that having stimulation modules and particularly with the incorporation of internal stimulation module parts and external stimulation module parts the device is particularly effective in providing a more effective application of a plurality of external stimulation without wires extending everywhere and with coordination with single user controller while still being networked for medical assessment and medical control.

It can therefore be that a user or under the guidance of your medical practitioner there is a selection of stimulator module or modules and these are installed into the user controller with an internal part in communication with an external part that is attached externally to the user when in use.

Looking at components in further detail:

1. Input Devices:

The input devices can be a sensor connectable external to the user for providing characteristics of the user that relate to the effective breathing of the user. Such sensors can be undertaking sensing of the ear, foot, or wrist or other extremity providing access to vital characteristics.

In the form of a Smart Ear device acting as an input device, it can send the following features and functions to the Health App:
i) Collected heart rate and blood oxygen (SpO₂) at predefined time intervals in real time to identify when the user is having a Sleep Apnea episode.
ii) And Utilizing gyroscope technology, accelerometer or similar identifies when the user is sleeping too long on their back.

The Smart Foot device, acting as an input device, it can send the following features and functions to the Health App:
i) Collect heart rate and blood oxygen (SpO₂) at predefined time intervals in real time to assist in identifying when the user is having a Sleep Apnea episode.

The Smart Band device, acting as an input device, can send the following features and functions to the Health App:
i) Collect blood pressure, heart rate and blood oxygen (SpO₂) at predefined time intervals in real time, indicating when the user is having a Sleep Apnea episode.
ii) Record sport data, health fitness activities and provide reminders.

2. Smart Devices and the Health App:

Various ‘smart’ devices can be used, which can connect with smart devices using smart technology including computers, tablets, phones, watches, wearable devices, or other Internet of Things (IOT) supporting Windows, Apple & Android. Data is collected from the smart input devices using Bluetooth, WiFi or other wireless technologies.

Users use the free Health App to setup and configure all input/output options to accommodate their specific requirements. The Health App manages all inputs and sends Output actions to the devices to inform the user.

The Input devices can also serve as the Output devices performing multiple functions.

3. Output Devices:

The Output devices receive notifications from the Health App via Bluetooth WiFi etc. for activating the external stimulator positionable relative to a user for providing an effective external stimulation option or external notification option to the user. The Output devices convert these incoming signals to specific actions and directly or indirectly informing or enticing or urging consciously, subconsciously or automatically the user to take preconfigured actions.

Clearly the most effective external stimulant is one that urges the user to automatically undertake an action to effect an alteration to the user's breathing.

There are various Output devices available to best accommodate the user's preferences and requirements. Output devices can be used to help manage sleep apnea, snoring and SIDS symptoms.

3A Output Devices—The TENS/EMS Device:

In using a TENS/EMS Device the device is fitted externally over the submental region directly under the user's chin. The device can be worn or fitted as part of a neck brace, chin strap, adhesive contact or similar.

In one form of TENS/EMS device a Head-Strap is fitted over the head, placing the “TENS” Stimulation-Device directly under the user's chin. The strap also holds the chin in a more optimal position, delivering additional benefits.

The “TENS”/EMS stimulation-device delivers the following to stimulate the submental area (tongue muscles, hypoglossal nerves) to move and help improve user's breathing by delivering a very small vibration and/or electrical stimulation via the TENS/EMS device that will:
notify the user to alter positions and/or take suitable steps
to improve their breathing capability and increase oxygen flow.
notify the user they are sleeping too long on their back,
prompting them to move to their side.

3B Output Devices—The Smart Ear:

The SmartEar is fitted into the user ear. This device is a multipurpose device, acting as an Input and Output device in one.

The SmartEar delivers the following to help move the tongue muscles and help improve users breathing by delivering a very small vibration that will:
a) notify the user to alter positions and/or take suitable steps to improve their breathing capability and increase oxygen flow.
b) notify the user via a Vibration or Sound they are sleeping too long on their back, prompting them to move to their side.
c) Enables you to receive/make calls, listen to music from the Smart device.
d) notify you when the smart device is low on battery.

3C Output Devices—The SmartBand:

The SmartBand is fitted onto the user wrist. This device is a multipurpose device, acting as an Input and Output device in one. It is also waterproof (IP67)

The SmartBand delivers the following to help move the tongue muscles and help improve users breathing by delivering a very small vibration that will:
a) notify the user to alter positions and/or take suitable steps to improve their breathing capability and increase oxygen flow.
b) notify the user via a Vibration or Sound they are sleeping too long on their back, prompting them to move to their side.

Other key functions include:
Enables you to receive/make calls, listen to music from the Smart device.
notify you when the smart device is low on battery.
Receive message from Facebook and Twitter
Select from 8 languages.
Monitor users sleep quality
Receive sport data and incoming reminders

3D Output Devices—The SmartFoot:

The SmartFoot is fitted over the user's foot. This device is a multipurpose device, acting as an Input and Output device in one.

The SmartFoot delivers the following to help move the tongue muscles and help improve users breathing by delivering a very small vibration that will:
a) notify the user to alter positions and/or take suitable steps to improve their breathing capability and increase oxygen flow.
b) notify the user via a Vibration or Sound they are sleeping too long on their back, prompting them to move to their side.
c) notify you when the smart device is low on battery.

3E Output Devices—TENS and EMS Technology

Hypoglossal nerve stimulation (HNS) has been undertaken primarily by invasive internal circumferential nerve cuff electrode, a stimulation lead and an implantable pulse generator.

There is use of TENS devices. TENS—stands for “Transcutaneous Electrical Nerve Stimulation”, a non-invasive drug free method used by physical therapists and prescribed by Doctors for over 30 years. TENS consists of a device that transmits low-level electrical impulses to the body. A mild electrical current travels through your skin and along your nerve fibres which may cause a warm, tingling sensation.

However, a problem had arisen from the previous observations, that such stimulating of a single protrusor muscle could result in the antagonistic activation of other neck and tongue muscles which could evoke an antagonistic effect on airway patency. However, stimulating the hypoglossal nerve could also lead to the stimulation of multiple tongue muscles, which could lead to a synergistic effect and a favorable effect.

By use of external stimulator there is a sifter more effective synergistic action on the tongue muscles and thereby improved breathing. TENS unit is designed to provide nerve stimulation, placing the electrode pads correctly on a muscle can cause a strong muscular contraction.

EMS stands for electrical muscle stimulation. So, you will be simulating a completely natural procedure. Since, for a muscle to move, it has to receive electrical stimulation via the nerve pathways. In an EMS treatment, this stimulation comes from a handy electrical pulsing device in the right strength and frequency via electrodes on the skin. By means of various current strengths, frequencies and intervals you can use your TENS-EMS device for selective muscle activation or relaxation.

For Correct Muscle Stimulation Through EMS, current devices allow you to choose between various programs and settings, Synchronous (S) and asynchronous (A): With synchronous programs, the stimulation is carried out simultaneously on all available channels, while with asynchronous programs this happens with a time delay. This, for example, allows a particularly thorough or a particularly gentle stimulation to be achieved. Clearly for external stimulation of the hypoglossal nerve at the rear of the oral cavity at the rear of the tongue a gentle but pulsed stimulation is effective.

Frequency is significant for most EMS applications and is given in Hertz (Hz). When choosing the frequency, you should take into account that there are individual differences.

Low frequencies (no higher than about 18 Hz) will mainly activate the slower reacting red muscle fibres. Higher frequencies between 30 and 50 Hz stimulate the fast-contracting white muscle fibres.

With frequencies of over 50 Hz, the muscle is deliberately overtaxed and can thus be forced into muscle hypertrophy (muscle build-up). The interval between the sessions must be correctly chosen so that the muscle has enough time to regenerate.

The pulse width or pulse duration is given in microseconds (μs). With longer pulses, the effect goes deeper and is mainly suited to larger muscles. For smaller muscles, the duration will remain below 200 μs. Some programs offer a varying pulse duration to stimulate the muscle even more intensively.

Cold muscles should never be put under full strain even with EMS. That is why modern EMS devices ensure that muscles are gently warmed and supplied with blood by pre-tensioning. For the untrained, the minimum time for this is 2 seconds.

The contraction time (ON) is chosen to be through numerous, relatively short (4-6 seconds) stimulations. Pauses are usually at least twice as long as the contraction time.

Modern TENS/EMS devices operate almost exclusively with biphasic pulses, which are gentler on the skin of the user. This means every current pulse is followed by a phase with a negative counter-oscillation below the zero line.

General settings can be:
Lower Frequency: Max. 15-18 Hz
Short Contraction Duration: 4-6 seconds
Short Pause Time: 3-6 seconds
Small Muscle: Low pulse width (50-100 μs)

Variable Usage

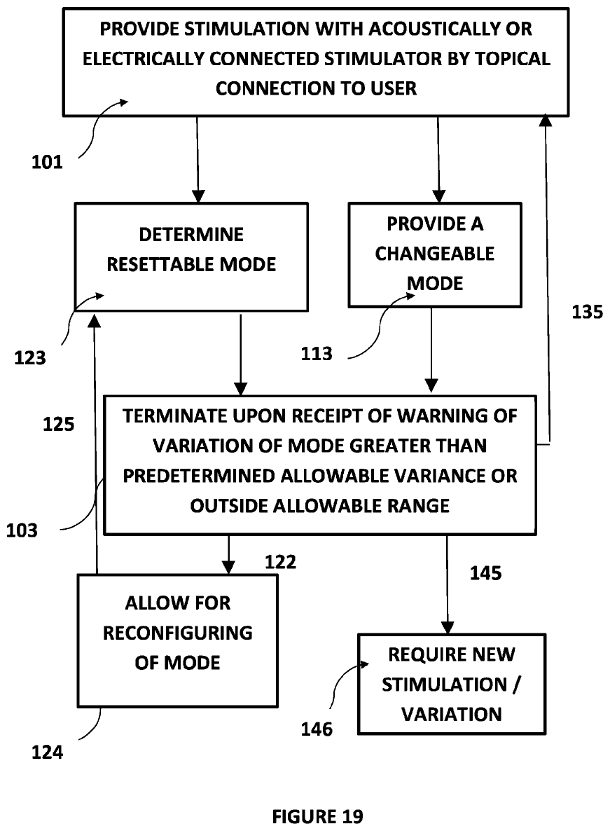

Referring to FIGS. 19 to 23 there is shown the self-modifying operation of the sensors and external stimulators by feedback and control in a variable system for aiding management of a breathing related disorder. This is particularly effective in ensuring that the body does not become acclimatised to the same stimulant and therefore becomes less effective.

Referring to FIG. 19, in one embodiment of a variable usage in which the predefined operative framework 15 is in receivable connection to the sensors 20 locatable on the user to receive information from the sensors about the sensed characteristic of a breathing related disorder and the predefined operative framework 15 has also operable connection to at least one external stimulator of a plurality of stimulators 40 positionable relative to a user for providing an effective external stimulation option to the user.

In operation of such variable usage, in step 101 there is provided stimulation acoustically or electrically or physically by an external stimulator 140 being positioned topically on the user.

In step 113 there is provided a changeable mode. This changeable mode can use a particularly effective mechanism of having a predefined operative framework with a plurality of external stimulators that are from more than one category. Therefore, the predefined operative framework identifying a plurality of stimulation options includes a plurality of categories or selection of at least one stimulation option in at least each of the plurality of categories by the user in their respective user input and wherein the categories of stimulation options include a plurality of:
a) Stimulation option category;
b) Adjusted stimulation option category; and
c) Adjusting form of stimulation from a stimulation in the Stimulation option category

The predefined operative framework identifying a plurality of stimulation options includes a plurality of categories includes a plurality of the stimulation option categories selected from:
a) Foot;
b) Wrist;
c) Ear;
d) Head;

The categories of stimulator can be selected from:
i) Acoustic which can give an audible alarm;
ii) Physical which can give a prod/vibration/displacement at the site of the hypoglossal nerves, the ears, toes, or fingers; or
iii) Electrical pulse which can give a short circuit to the hypoglossal nerves to activate tongue retraction and to help improve breathing.

In providing the changeable mode step 113 there is the operation of the mode until step 103 of determining and warning of the variation of the mode of the sensor 20 and/or external stimulator 140 being greater than predetermined allowable variance or outside allowable range. This is further described later with reference to FIGS. 10 to 13.

From the changeable mode step 113 being undertaken there are the two options of step 145 of a new mode or step 124 of altering of the present mode.

In step 145 the mode of external stimulator moves to requiring a new stimulation or variation of step 146. In this form, a different external stimulator 140 can be connected to the predefined operative framework 15. This can be by purchasing a different device or relocating to a different body part. Step 135 requires the return to initial step 101 after the new external stimulator 140 is connected, and set-up through the controls 51, 52 and 53.

In the option of step 122 of moving to the step 124 of the adjusting form of stimulation. This adjustment step requites a determination step 123 of how to reset the mode to a new operational position with the same external stimulator but with a different operative mode from a stimulation in the Stimulation option category is selected from:
a) Variable selected frequency,
b) magnitude,
c) period,
d) symmetry of stimulation.

Looking in more detail at the step 103 there can be operative self-assessment and adjustment of the sensors 20 in accordance with their operative conditions and predetermined medical operation conditions and personal preference operative conditions by the communication with the user control 51, set-up control 52 and medical control 53. There also can be operative self-assessment and adjustment of the external stimulators 40 in accordance with their operative conditions and predetermined medical operation conditions and personal preference operative conditions by the communication with the user control 51, set-up control 52 and medical control 53.

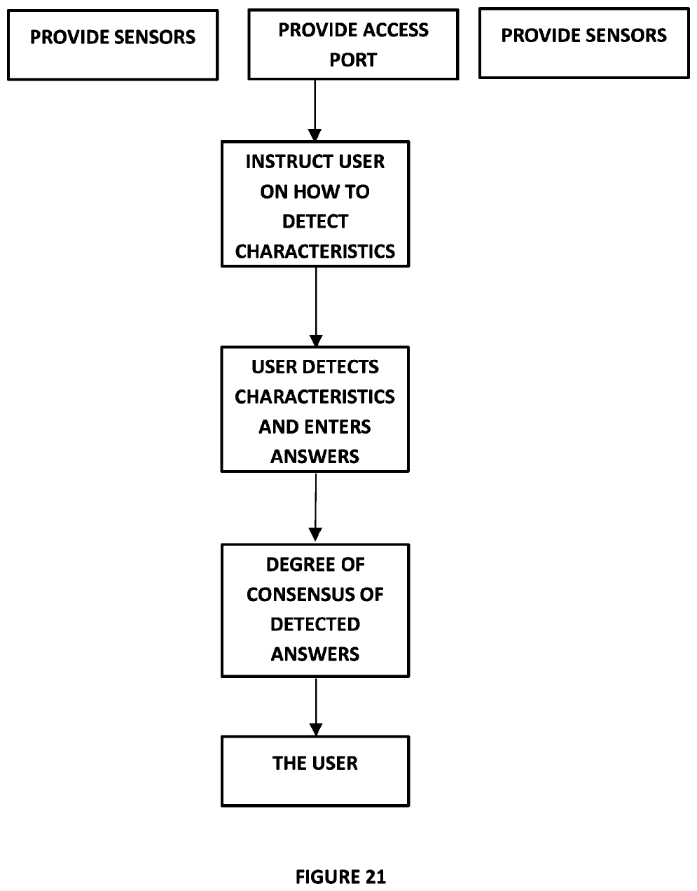

Referring to FIGS. 20 and 21 there can be a particular sensor 20 that can operate between A and B. However, the control means provides an operative range between D and H that is predetermined so that it would provide the effective sensing by the sensor of a characteristic of the user's breathing and sensing of the effects an alteration to the user's breathing. The operation of the sensor can therefore be at E which is within the range of D to H on the A-B scale. However, by feedback of the sensors it can be determined if the effectiveness has slipped and needs change to G to re-enhance the effectiveness. Still further if the feedback shows the stimulator falling outside the effective range of D to H then a signal can be sent to the mode effectiveness warning. This can ensure that the variation of the effectiveness in the predefined sensor is immediately instigated. This can allow for change of sensor or for a totally different operating sensing range from A-B is chosen or a change in pick-up signal or change in intensity. It can also allow change of category of sensor.

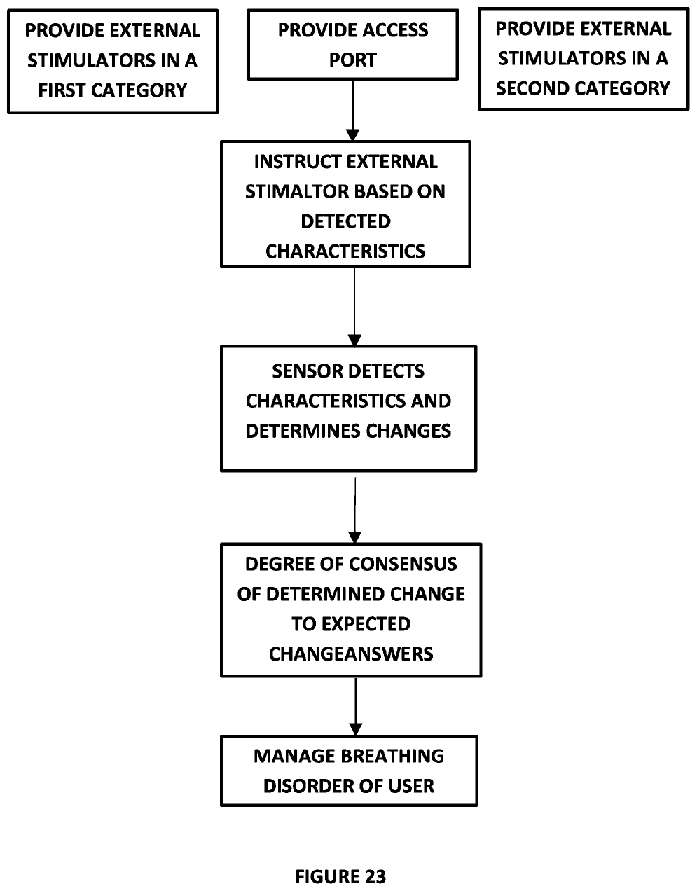

Referring to FIGS. 22 and 23 there can be a particular external stimulator 140 that can operate between X and Y. However, the control means provides an operative range between M and N that is predetermined so that it would provide the effective external stimulation by the at least one external stimulator effects an alteration to the user's breathing. The operation of the stimulator can therefore be at P which is within the range of M to N on the X-Y scale. However, by feedback of the sensors it can be determined if the effectiveness has slipped and needs change to Q to re-enhance the effectiveness. Still further if the feedback shows the stimulator falling outside the effective range of M to N then a signal can be sent to the mode effectiveness warning. This can ensure that the variation of the selection in the predefined operative framework is immediately instigated. This can allow for change of mode of stimulator such that a totally different operating range from X-Y is chosen or a change in signal or change in intensity. It can also allow change of category of stimulator.

It can be seen that the system is built in order to overcome the propensity for the user to be de-sensitised and acclimatized to the external stimulant. Further the sensor effectiveness might change due to the sensed characteristic no longer being related as precisely. Recalibration can be automatically achieved by the reassessment of the sensed characteristic and its meaning for the particular user at that particular time by the feedback of sensing and stimulation and further sensing.

### Example—Scenario A

In its simplest form, the Apnea user would go to bed along with the usual anxieties associated with anticipated breathing difficulties or in some cases complete lack of oxygen and the dreaded awakening or choking symptoms. These factors contribute in addition to the Apnea side effects, which include apprehension, anxiety, stress and insomnia, which in turn contributes to other health issues.

Whilst sleeping the simplest form of the management App would involve the Apnea recipient (user) to wear an input device on a preferred part of their body, based on personal preference e.g. wrist, foot, ear, etc.

This input device would take readings of the users' Oxygen levels via Oximetry and Pulse recordings.

The Input Device(s) measure concurrently and detect both the Oxygen changes and pulse level changes and prepares this data to be sent to the Apnea Management System.

The Apnea Management System receives data from the Input Device(s) via Wireless* technology and identifies pre-configured reductions in oxygen level with concurrent increase in pulse level, identifying the onset of symptoms which could contribute to an apnea episode and alert the user based on their settings.

The Apnea Management System would communicate via Wireless technology with the Output Device(s) via audible and/or vibrational methods. This alerting method will notify the user of a possible apnea episode and based on their settings, they can simply be gently notified (partially arouse). The alerting will increase in severity if user does not move and/or symptoms increase in severity.

The intended outcome here is to inform the user prior to experiencing the full onset of apnea symptoms to prevent short term (snoring effects) and long-term effects. (Wireless*=Wifi, Bluetooth and/or similar technologies) Note: The system can use both TENS and/or EMS

### Example—Scenario B

Oxygen deprivation onset sets in when it reduces from 98%+ to 50-60% and increasing Pulse rate above typical resting levels. These are usually the two main indicators of Apnea that we can focus on enabling these readings to be communicated via Bluetooth to the Apnea Management System and subsequent Output management devices in either scenario A or B.

Whilst the sleeping scenario is the same as the above in terms of monitoring and the function of the input device(s), however in this scenario the technology used for the output device in this scenario will communicate via wireless with an output device located on the user's chin.

This output device is based on TENS technology and will be housed within a chin strap or self-adhesive, which would arouse the Hypoglossal region (nerve stimulation) to such an extent that the tongue muscle remains in a non-collapsed state averting apnea type symptoms, etc. These setting are fully controllable and customised by the user to ensure the TENS levels are set to an effective level specifically suited to them, which contributes to minimum sleep disruption and maximise apnea management.

As the human brain has the ability to eventually recognise nerve stimulus settings and could ignore the same stimulation over a period of usage. To ensure ongoing effectiveness the Apnea Management System will allow users to randomly select various ranges and depths of TENS stimulus.

The TENS output device also serves as a therapeutic benefit in allowing the user to enhance these muscles due to the ongoing stimulation. Effectively strengthening the muscles which could contribute to reduced apnea episodes.

The Apnea Management System will allow the user the option of selecting their preferred levels of nerve/muscle stimulation so as to fine tune minimum disruption from sound sleep and maximum management of Apnea symptoms and long-term health issues.

The Apnea Management System will monitor other aspects of their Health, enabling the user to record information which they can either use personally or provide to third parties. This data will also help their care practitioners to develop improved care plans. Examples include, ongoing Oxygen levels, pulse rates, steps, calories burnt. The user will also be able to enter other health data which will enable them and their doctor to correlate other health data in conjunctions with the above data, to provide a holistic assessment. This will include the ability to enter their weight, BMI, blood sugar level (Diabetics), carbs consumed, cholesterol, etc The Apnea Management System will have content feeds from various health sources and forums with social media integration.

The Apnea Management System will have an Alert Management System. This enables users to configure notifications to be sent to their partners, personal carers or nurses in the event their monitoring detects critical stages where the user has not responded and/or moved. This type of notification is useful if users have
Their partner sleeping in another room.
Their family member sleeping in another room.
monitoring by a nurse within a hospital.
monitoring by a loved one remotely.
Enabling users to configure reports to be sent to their doctors (or care givers) to manage their progress.
Enabling users to be informed that the Apnea Management System is running at full capacity with high regularity, recommending they consult their doctor for a CPAP assessment.

### Example—Scenario C

In a multiple sensor form there can be input sensors of:
1. O2 Oxygen. (Levels below 92% Oxygen in your blood is a sign of a breathing problem.
2. Pulse/Heart Rate
3. Blood pressure
4. Geo-Positioning (Upright, Laying down, Moving Activity)
5. Temperature
6. Diaphragm movement.
7. Steps & Activity & Floors Climbed
8. Calories Burned
9. Sleep Tracking
10. Sleep Stages (Light, Deep, REM)

In Multiple sensors the approach can be:

| | | | |
|---|---|---|---|
| Typical User | 1. | O2 oxygen sensor | Most effective with two primary |
| | 2. | Pulse/heart rate | sensors but matched with secondary |
| | | sensor | sensor with programmable weighting |
| | 4. | Geo Positioning | or selective single sensor trigger |
| | | Sensor | points as effective collaboration of |
| | | | multiple single sensor trigger points |
| Lone Sensor User | 1. | O2 oxygen sensor | Effective single sensor that is non- |
| | | | invasive but is a primary sensor. |
| Simple watch | 2. | Pulse/heart rate | Primary sensor and secondary |
| Based Sensors | | sensor | sensor in readily created user |
| | | [001]Geo Positioning | controllers such as in digital watches |
| | | Sensor | |
| Unstable O2 | 3. | Blood Pressure | Primary and secondary sensors that |
| detection User | | Diaphragm | overcome particular problems of |
| | | movement | particular users that have problems |
| | | | with the effectiveness of above |
| | | | systems working for that user. |

In Multiple sensors the approach can be The Apnea App system will receive input data feed, in real time, from users watch device and other suitable devices, based on features and functions available to that device the user wants to utilise with their Apnea App.

Input functions 1 to 6 will contribute to an OUTPUT action to support the users Apnea profile. Input functions 7 to 10 will contribute to provide analysis into Users Dashboard in the Apnea App, providing analysis of these inputs, correlation of inputs 1 to 7 on how they may have varied and correlation to the Questionnaire responses they entered.

In this scenario, the user turns off Inputs 3 and 5, as they do not have Blood pressure issues and exercises in the evening regularly which could provide adverse temperature readings. User has also disables Input 6 as they do not have that input sensor.

Monitoring actions include:
The Apnea App will monitor inputs 9 & 10 and track till user falls asleep.
As User has identified in their questionnaire they are experiencing significant Apnea symptoms and difficult to sleep, they have further set Input 10 configuration to the ‘Deep’ level, to start taking Apnea preventative actions at this level as they don't easily reach the “REM” sleep stage.
The Apnea App with monitor input 4 to ensure they are not upright and laying down. This input works in parallel to input 9 & 10 to deliver accurate assessments.
The Apnea App with monitor Inputs 1 & 2 in line with user settings.

### Low-Level-Output Condition:

Once Inputs 1 & 2 detect and assess the criteria and configurations meets this condition, it will instigate and trigger the configured Output condition. Assess how much below trigger point did level go, how rapid was the decline and if its stabilised at the low level.

The Apnea App will monitor for re-occurrence event within a time cycle, as defined by user, and re-trigger Output condition. The Apnea App will monitor for frequency cycles on this condition and based on configuration by user, escalate up to a Mid-Level-Output condition. If configured by the user, a Low-Level-Output can include a TENS pulse (100 Hz, 200 ms) directly over the hypoglossal nerve.

### Mid-Level-Output Condition:

Assess volume of Low-Level-Output conditions triggered during this sleep cycle, and/or, assess configured conditions requiring a Mid-Level-Output condition output to be triggered. (Note: A Mid-Level-Output is at a higher level than a Low-Level-Output.)

The Apnea App will monitor for re-occurrence event within a time cycle, as defined by user, and re-trigger Output condition. The Apnea App will monitor for frequency cycles on this condition and based on configuration by user, escalate up to a High-Level-Output condition. If configured by the user, a Mid-Level-Output condition can include a TENS multiple pulse (100 Hz, 225 ms) directly over the hypoglossal nerve.

### High-Level-Output Condition:

Assess volume of Mid-Level-Output conditions triggered during this sleep cycle, and/or, assess configured conditions requiring a High-Level-Output condition output to be triggered. (Note: A High-Level-Output is at a higher level than a Mid-Level-Output.)

The Apnea App will monitor for re-occurrence event within a time cycle, as defined by user, and re-trigger Output condition. The Apnea App will monitor for frequency cycles on this condition and based on configuration by user, escalate final Duress-Output to awaken the user. The Duress-Output is configured by user which can include one or more of the following:
Continuously vibrate Users wrist device and/or other configured device(s)
Continuously produce an audible tone on Users wrist device and/or other configured device(s)
Call users preconfigured caller list, family, medical assist, etc.
Send SMS/email alerts and/or other similar event notifications to preconfigured parties who may be setup to monitor user, which may have Dashboard setup.

If configured by the user, a High-Level-Output can include a TENS multiple pulse and combination (100 Hz, 250 ms) directly over the hypoglossal nerve.

Based on User configuration and amount of Apnea App stimulations to help prevent/manage Apnea episodes, the system will inform user to seek alternative (CPAP) options via appropriate consultation. Record all output actions taken for future review.

In this scenario, the user is only using Inputs 1 (O2 Oxygen) and 4 (Geo-Positioning). The Apnea App will monitor inputs 9 & 10 and tack till user falls asleep. Once user is sleeping the Apnea App will monitor Input 1 (O2 Oxygen) level and when it drops below the configured value of 85%, it will then check input 4 (Geo-Positioning)

Low-Level-Output condition: If the User is laying on their back, they will be notified via the configured Output settings to move and lay on their side.

Mid-Level-Output condition: Not enabled, use has disabled.

High-Level-Output condition: Enabled to wake the user if 3×Low-Level-Input conditions occur within 5 min and there is no action.

In this scenario, the user can use Key Output Devices including:
Wrist strap/watch
Other ear, nose, audio prompts, neck, arm, and other body part devices.
Technical devices able to integrate with Apnea App System
Smart phones, watches, tablets, laptops, notepads, computers, smart speakers capable of providing configurable/automated responses to user, etc.
Wire or wireless/Bluetooth technology or similar.

Key OUTPUT functions are provide the Output functions, which are configurable key output devices that can be used in any combination by the user as part of their management plan, which is fully configurable by the user.

Output Devices produce various actions/output, which can include vibration, audible sounds, other configurable Output Device capability, notifications (e.g. emails, calls, push-notifications, social media integration, hospital/medical systems, etc.) stimulation (e.g. TENs, electrical impulse to key areas, including the Hypoglossal Nerve, Neck or other approved areas)

The Apnea App HealthTrack Dashboard will correlate Apnea triggers, Output events and correlate all inputs to display a Dashboard that representation of all factors. For example, the User will be able to see in the Dashboard that since they have lost xKgs in weight and walking 10,000 steps per day, their Apnea episode volume/count is trending down and/or the volume of High-Level-Output conditions is reduced by 20%. The HealthTrack Dashboard will also represent the opposite outcomes if the data supports that.

The Apnea App can determine Low (Mild), Med (Moderate), or High (Severe), depending on the number of times in an hour the users breathing stops (Apnea) or becomes very shallow (hypopnea).
Apnea episodes may occur from 5 to 100 times an hour.
More than 5 Apnea's per hour is abnormal.
5-10 per hours can be considered Low (Mild) sleep Apnea.
10-30 per hours can be considered Med (Moderate) sleep Apnea.
More than 30-40 per hour can be considered High (Severe) sleep Apnea.

The Advanced Apnea Management System will be used by hospitals, retirement centres, family members, who have the need to manage/monitor multiple people using their own Apnea Management System. This is like a multi-user management system, which can see vital signs, as configured, of multiple people, simultaneously.

All hardware/technologies required for the Input and Output devices currently exist. The Apnea Management System is the unique method that brings all the processes, functions and algorithms together to manage the devices within the Apnea, Snoring and SIDS arenas.

The aim is to provide a method of monitoring and management to reduce Apnea episodes and symptoms, assisting people to reduce the effects of Apnea and delay the need to use a CPAP machine in the first instance of their treatment plan.

### Interpretation

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In the description provided herein, numerous specific details are set forth. It is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. The invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to the health industry and self-help health industry industries and in particular to the field of treatment, management and pre-management against disorders such as sleep apnea or snoring or sudden infant syndrome.

The claims defining the invention are as follows:

1. A system for aiding early detection and management of a breathing related disorder comprising:
a. A user controller
b. At least one sensor connectable to the user controller for sensing and providing sensed data of at least one characteristic relevant to a breathing related disorder;
c. A predefined operative framework in the user controller having connection for receiving the sensed data from the at least one sensor;
d. at least one external stimulator positionable relative to a user and operatively connectable with the predefined operative framework for receiving operative instruction and providing an effective external stimulation to the user;
wherein the system aids early detection and management of a breathing related disorder by an effective sensing by the at least one sensor.

2. A system for aiding early detection and management of breathing related disorders according to claim 1 wherein the breathing related disorders include one or more of:
a. Sleep apnea
b. Snoring
c. SIDS (Sudden Infant Death Syndrome)

3. A system for aiding early detection and management of breathing related disorders according to claim 1 or 2 including a control means for receiving to the predefined operative framework a predefined effective operative range of the at least one external sensor;
wherein the predefined effective treatment range and control aids early detection and management of a breathing related disorder by an effective sensing by the at least one sensor.

4. A system for aiding early detection and management of breathing related disorders according to claim 1 or 2 including
a. a plurality of the at least one external sensor and
b. a control means for receiving to the predefined operative framework a predefined effective operative range of each of the plurality of the at least one external sensor;
wherein sensing by at least two of the plurality of the at least one external sensor within the predefined effective treatment range and control aids early detection and management of a breathing related disorder by an effective sensing by the at least one sensor.

5. A system for aiding early detection and management of breathing related disorders according to claim 5 wherein the control means includes
a. a collaborator
b. and each sensor receives sensed data and assesses if a predefined trigger point has been sensed and sends identification of single sensor trigger point to the collaborator
c. the collaborator receiving and undertaking a trigger analysis including reviewing multiple sensors
i. and if multiple single sensor trigger point from multiple sensors have been received within a predefined time period creating a trigger actuation and outputting trigger output to operative framework to instigate external stimulation;
ii. and if multiple single sensor trigger point from multiple sensors have not been received within a predefined time period return to further sensing and further trigger analysis
wherein the recordal of a single sensor trigger point of one sensor does not initiate trigger actuation unless supported by a single sensor trigger point of another sensor.

6. A system for aiding early detection and management of breathing related disorders according to claim 1 or 2 wherein the at least one external sensor is selected from a sensor positionable to sense at any one or more of the following external body sensor locations of:
a. Head
b. throat
c. nose
d. mouth
e. Ear
f. Wrist
g. Finger
h. Foot/limb

7. A system for aiding early detection and management of breathing related disorders according to claim 6 wherein the at least one external sensor is selected from a sensor to sense according to at least one of the following modes of:
a. Pulse/heart rate
b. Oximetry/O2 levels
c. Temperature
d. Body movement
e. breath

8. A system for aiding early detection and management of breathing related disorders according to claim 1 or 2 including
a. a control means for providing to the predefined operative framework a predefined effective operative range of the at least one external stimulator;
wherein the predefined effective treatment range and control aids early detection and management of a breathing related disorder by an effective external stimulation.

9. A system for aiding early detection and management of breathing related disorders according to claim 1 wherein the effective external stimulation by the at least one external stimulator effects an alteration to the users breathing.

10. A system for aiding early detection and management of breathing related disorders according to claim 3 wherein the effective external stimulation by the at least one external stimulator indirectly effects an alteration to the users breathing.

11. A system for aiding early detection and management of breathing related disorders according to claim 4 wherein the effective external stimulation by the at least one external stimulator indirectly effecting an alteration to the users breathing is by notification to the user.

12. A system for aiding early detection and management of breathing related disorders according to claim 3 wherein the effective external stimulation by the at least one external stimulator substantially directly effects an alteration to the users breathing.

13. A system for aiding early detection and management of breathing related disorders according to claim 6 wherein the effective external stimulation by the at least one external stimulator directly effecting an alteration to the users breathing is selected from one or more of:
a. an external stimulator locatable on or near the ear of the user providing acoustic output for stimulation to the user,
b. an external stimulator locatable on or near the eye of the user providing light output for stimulation to the user
c. an external stimulator locatable under chin for vibration stimulation to the user,
d. an external stimulator locatable on the head for external stimulation to the user,
e. an external stimulator locatable on the finger for tactile stimulation to the user,
f. an external stimulator using TENS (Transcutaneous Electrical Nerve Stimulation) or EMS (Electrical Muscle Stimulation) or both locatable to provide for stimulation to the user, or
g. an external stimulator locatable on the foot for stimulation to the user.
wherein the external stimulators can operate in a mode that directly affects a user to improve the breathing of the user.

14. A system for aiding early detection and management of breathing related disorders according to any one of claims 1 to 7, wherein the communication between sensors, control and at least one external stimulator is wireless.

15. A system for aiding early detection and management of breathing related disorders according to claim 1 or 2 wherein the user controller includes connection with one or more input selectable sensor modules providing the at least one sensor.

16. A system for aiding early detection and management of breathing related disorders according to claim 15 wherein the one or more input selectable modules includes a selected sensor internal module part for connection with the user controller and an external sensor module part for location on an external body location and for providing sensed data to the selected sensor internal module part.

17. A system for aiding early detection and management of breathing related disorders according to claim 16 wherein the external sensor module part is positionable to sense at at least one or more of the following external body sensor locations of:
a. Head
b. throat
c. nose
d. mouth
e. Ear
f. Wrist
g. Finger
h. Foot/limb

18. A system for aiding early detection and management of breathing related disorders according to claim 16 or 17 wherein the external sensor module part is selected to sense according to at least one of the following modes of:
a. Pulse/heart rate
b. Oximetry/02 levels
c. Temperature
d. Body movement
e. breath

19. A system for aiding early detection and management of breathing related disorders according to claim 1 or 2 wherein the user controller includes connection with one or more output selectable stimulator modules providing the at least one external stimulator.

20. A system for aiding early detection and management of breathing related disorders according to claim 20 wherein the one or more output selectable stimulator modules includes a selected stimulator internal module part for connection with the user controller and an external stimulator module part for location on an external body location and for providing stimulation to the selected stimulator external module part.

21. A system for aiding early detection and management of breathing related disorders according to claim 21 wherein the external stimulator module part is positionable to stimulate at at least one or more of the following external body sensor locations of:
a. Head
b. throat
c. nose
d. mouth
e. Ear
f. Wrist
g. Finger
h. foot

22. A system for aiding early detection and management of breathing related disorders according to claim 20 or 21 wherein the external stimulator module part is selected to sense according to at least one of the following modes of:
a. Electrical pulse TENS/EMS
b. Sound/Audio
c. Touch/Physical
d. Vibration
e. Light

23. A system for aiding management of a breathing related disorder comprising:
a. At least one sensor for sensing at least one characteristic of a breathing related disorder;
b. An access port for upload of details of the at least one characteristic of a breathing related disorder from the sensor to an online computerised means;
c. A receiver port, for download from the online computerised means, which is adapted for receiving computerised instructions according to a predefined effective treatment range in managing control of a breathing related disorder;
d. At least one topical stimulator positionable on a user for providing an effective stimulation option to the user and including a receiver for receiving electronic control instructions;
e. At least one input device for allowing a user to predefine at least one user particular stimulation output and provide for upload to the access port;
f. At least one display device for allowing display of an external stimulation output to the user to provide a confirmed user defined management control of the breathing related disorder application;
g. At least one transmitter for transmitting each user particular input of the plurality of users to the access port of the online means for upload to the online computerised means; and
h. At least one receiver for transmitting for receiving by at least one of the at least one display device for allowing display of the respective confirmed user defined breathing related disorder application of each user;
i. wherein the plurality of user particular inputs defines the predefined effective treatment range and control of a breathing related disorder and the selection of an effective stimulation option defines the chosen effective option and is provided to the at least one display device.

24. A system for aiding early detection and management of breathing related disorders according to claim 9 wherein the breathing related disorders include one or more of:
a. Sleep apnea
b. Snoring
c. SIDS (Sudden Infant Death Syndrome)

25. A system for aiding early detection and management of breathing related disorders according to claim 9 wherein the at least one input device provides a predefined operative framework identifying a plurality of stimulation options for selection by the user in their respective user particular input wherein a selection of an effective stimulation option from each category matching a user particular input of the same stimulation options in each category defines the chosen effective option.

26. A system for aiding early detection and management of breathing related disorders according to claim 9 wherein the predefined operative framework identifying a plurality of stimulation options includes a plurality of categories or selection of at least one stimulation option in at least each of the plurality of categories by the user in their respective user particular input.

27. A system for aiding early detection and management of breathing related disorders according to claim 9 wherein the predefined operative framework identifying a plurality of stimulation options includes a plurality of categories or selection of at least one stimulation option in at least each of the plurality of categories by the user in their respective user particular input and wherein there are at least 2 categories of stimulation options and wherein a selection of an effective stimulation option from each category matching a user particular input of the same stimulation options in each category defines the chosen effective option.

28. A system for aiding early detection and management of breathing related disorders according to claim 9 wherein the predefined operative framework identifying a plurality of stimulation options includes a plurality of categories or selection of at least one stimulation option in at least each of the plurality of categories by the user in their respective user particular input and wherein the categories of stimulation options include a plurality of:
a. Stimulation option category;
b. Adjusted stimulation option category; and
c. Adjusting form of stimulation from a stimulation in the Stimulation option category

29. A system for aiding early detection and management of breathing related disorders according to claim 9 wherein the predefined operative framework identifying a plurality of stimulation options includes a plurality of categories or selection of at least one stimulation option in at least each of the plurality of categories by the user in their respective user particular input and wherein there are at least 5 categories of stimulation options in the stimulation option category and wherein a selection of an effective stimulation option from each category matching a user particular input of the same stimulation options in each category defines the chosen effective option.

30. A system for aiding early detection and management of breathing related disorders according to claim 9 wherein the adjusting form of stimulation from a stimulation in the Stimulation option category is selected from:
a. Acoustic
b. Physical
c. Electrical pulse

31. A system for aiding early detection and management of breathing related disorders according to claim 9 wherein the adjusting form of stimulation in the same stimulation option category is selected from:
a. Variable selected frequency,
b. Change of magnitude,
c. Change of period,
d. Change of symmetry of stimulation.

32. A system for aiding early detection and management of breathing related disorders according to claim 9 wherein the predefined operative framework identifying a plurality of stimulation options includes a plurality of categories includes a plurality of the stimulation option categories selected from:
a. Foot;
b. Wrist;
c. Ear;
d. Head;

33. A system for aiding early detection and management of breathing related disorders according to claim 9 wherein the cost of a confirmed user defined breathing related disorder application is automatically determined by relevance to the selection of stimulation options of the user in the confirmed user defined breathing related disorder application and the plurality of user particular inputs defining the predefined effective treatment range and control of the breathing related disorder.

34. A method of automatically creating and running a freeform breathing related disorder using a computerised system including the steps of:
a. Providing a predefined operative framework identifying a plurality of stimulation options for selection by the user in their respective user particular input wherein a selection of an effective stimulation option from each category matching a user particular input of the same stimulation options in each category defines the chosen effective option
b. Receiving the user defined breathing related disorder application from a user over a digital communication system connected to an access port for upload to an online computerised means adapted for following computerised instructions according to a predefined effective treatment range and control of a breathing related disorder;
c. selecting an effective stimulation option according to the predefined operative framework;
d. comparing the plurality of effective stimulation options from each category to each of confirmed user defined breathing related disorder application wherein a selection of an effective stimulation option from each category matching a user particular input of the same stimulation options in each category defines the chosen effective option.

35. A method according to claim 20 wherein the predefined operative framework identifying a plurality of stimulation options includes a plurality of categories or selection of at least one stimulation option in at least each of the plurality of categories by the user in their respective user particular input and wherein there are at least 5 categories of stimulation options and wherein a selection of an effective stimulation option from each category matching a user particular input of the same stimulation options in each category defines the chosen effective option.

36. A method according to claim 20 wherein the predefined operative framework identifying a plurality of stimulation options includes a plurality of categories or selection of at least one stimulation option in at least each of the plurality of categories by the user in their respective user particular input and wherein there are at least 8 categories of stimulation options and wherein a selection of a effective stimulation option from each category matching a user particular input of the same stimulation options in each category defines the chosen effective option.

37. A method according to claim 20 wherein the predefined operative framework identifying a plurality of stimulation options includes a plurality of categories or selection of at least one stimulation option in at least each of the plurality of categories by the user in their respective user input and wherein the categories of stimulation options include a plurality of:
a. Stimulation option category;
b. Adjusted stimulation option category; and
c. Adjusting form of stimulation from a stimulation in the Stimulation option category

38. A method according to claim 20 further providing a plurality of categories for defining at least one selection must be made in each category;

39. A method according to claim 20 further Providing a plurality of stimulation options each allocated to one only of the plurality of categories;

40. A method according to claim 20 wherein the adjusting form of stimulation from a stimulation in the Stimulation option category is selected from:
a. Variable selected frequency,
b. magnitude,
c. period,
d. symmetry of stimulation.

41. A method according to claim 20 wherein the predefined operative framework identifying a plurality of stimulation options includes a plurality of categories or selection of at least one stimulation option in at least each of the plurality of categories by the user in their respective user particular input and wherein there are at least 5 categories of stimulation options in the stimulation option category and wherein a selection of a effective stimulation option from each category matching a user particular input of the same stimulation options in each category defines the chosen effective option.

42. A method according to claim 20 wherein the predefined operative framework identifying a plurality of stimulation options includes a plurality of categories includes a plurality of the stimulation option categories selected from:
a. Foot;
b. Wrist;
c. Ear;
d. Head;

43. A method according to claim 20 wherein the cost of a confirmed user defined breathing related disorder application is automatically determined by relevance to the selection of stimulation options of the user in the confirmed user defined breathing related disorder application and the plurality of user particular inputs defining the predefined effective treatment range and control of the breathing related disorder.
